(19) United States
(12) Patent Application Publication
(10) Pub. No.: US 2014/0079747 A1
(43) Pub. Date: Mar. 20, 2014

(54) Spray Drying Microcapsules

(71) Applicant: The Procter & Gamble Company, Cincinnati, OH (US)

(72) Inventors: Jiten Odhavji DIHORA, Liberty Township, OH (US); Jonathan Robert CETTI, Mason, OH (US); Steven Edward WITT, Morrow, OH (US); Jianjun Justin LI, West Chester, OH (US)

(73) Assignee: The Procter & Gamble Company, Cincinnati, OH (US)

(21) Appl. No.: 14/032,859

(22) Filed: Sep. 20, 2013

Related U.S. Application Data
(60) Provisional application No. 61/703,616, filed on Sep. 20, 2012.

(51) Int. Cl. A61K 8/02; A61Q 5/02; C11D 3/50; A61Q 15/00; A61K 8/28; C11B 9/00

(52) U.S. Cl. CPC A61K 8/0245; A61K 8/28; C11B 9/00; C11D 3/505; A61Q 15/00; A61Q 5/02
USPC 424/401; 512/4; 510/276; 510/120

(57) ABSTRACT

Spray drying microcapsules with particulates, the microcapsules that result from such spray drying, and compositions and methods of making said compositions including the spray-dried microcapsules.

### FIELD

The present disclosure generally relates to compositions and microcapsules, and specifically relates to spray-drying microcapsules, and the resulting spray-dried microcapsules being coated with particulates.

### BACKGROUND

Many products include microcapsules. A microcapsule is a micro-sized structure. Many microcapsules have an overall size that is measured in micrometers.

A microcapsule typically has a shell that encapsulates a core material. Microcapsules can be used to encapsulate various substances. For example, a microcapsule can be used to encapsulate perfume.

The shell of a microcapsule can be made from various materials. Some shell materials are meltable. A meltable material is a material with a low glass transition temperature. For example, a shell can be made from polyacrylate, which may or may not be a meltable material. Herein, a reference to a meltable microcapsule refers to a microcapsule with a meltable shell.

A microcapsule is useful for isolating the core material from its surroundings, until the encapsulated material is ready to be released. Depending on the kind of microcapsule, the core material can be released in various ways. One kind of microcapsule is a friable microcapsule. A friable microcapsule is configured to release its core substance when its shell is ruptured. The rupture can be caused by forces applied to the shell.

Microcapsules can be provided in various forms. For example, microcapsules can be provided in a liquid medium such as an aqueous slurry. To obtain the microcapsules from the slurry, the slurry can be dehydrated. For example, the slurry can be dehydrated with a spray-drying process. A spray-drying process disperses a liquid into small droplets. The droplets may be carried with a working fluid (such as air) that moves inside of a drying chamber. The working fluid (which may be heated) may cause the liquid to evaporate, leaving behind the dried microcapsules. The dried microcapsules can then be collected from the process equipment. Unfortunately, the spray-drying process can present difficulties to some kinds of microcapsules.

During spray drying, the hard impacts of the microcapsules can result in a problematic condition. As the microcapsules move around inside of the drying chamber, the microcapsules tend to impact the inside surfaces of the chamber and other microcapsules. For friable microcapsules, these impacts can cause their shells to rupture prematurely. Those ruptured microcapsules are no longer useful for isolating their cores from their surroundings as some or all of the core material may no longer be encapsulated by the shell. If a significant percentage of microcapsules are ruptured during the spray-drying process, then the process may not be commercially viable.

One approach to addressing such premature ruptures is to coat the microcapsules with a film. For example, the outer shell of a microcapsule can be coated with a soluble film. However, a microcapsule that is coated with a film may require a more complex way to release the core. For example, a microcapsule that is coated with a soluble film may first require a step of dissolving of the coating and followed by a second step involving the application of forces to rupture the shell in order to release the core material. This additional complexity may be undesirable for certain applications.

During spray drying, another difficult process condition is high heat. When the working fluid is heated, the microcapsules also heat up. For microcapsules with meltable shells, this heating can cause their shells to become sticky. The heated microcapsules may tend to stick to the inside surfaces of the drying chamber. The microcapsules that are stuck to these surfaces often cannot be collected from the process equipment with ease. If a significant percentage of the microcapsules cannot be collected from the spray-drying process, then the process may not be commercially viable for certain applications like the production of compositions including microcapsules.

Also, meltable microcapsules tend to clump together in the heat. The microcapsules that clump together can be difficult to further process, such as by incorporating the microcapsules into a finished product. If a significant percentage of spray-dried microcapsules cannot be used in a finished product, then the process may not be commercially viable for certain applications like the production of compositions including microcapsules.

### SUMMARY

A method of making a composition may comprise spray-drying a plurality of microcapsules, the microcapsules comprising a core material and a shell encapsulating the core material, with particulates to form spray-dried microcapsules, the spray-dried microcapsules comprising the core material and the shell encapsulating the core material, and adding a plurality of the spray-dried microcapsules to an adjunct ingredient to form a composition; wherein the spray-dried microcapsules are coated with the particulates.

The composition may comprise a plurality microcapsules comprising a core material and a shell encapsulating the core material; and an adjunct ingredient; and a median volume-weighted average particle size of from 3 micrometers to 25 micrometers; wherein the shell of the microcapsule is coated with particulates.

The microcapsules may comprise a core material and a shell encapsulating the core material; and a median volume-weighted average particle size of from 3 micrometers to 25 micrometers; wherein the shell of the microcapsules is coated with particulates.

A method of spray-drying the microcapsules may comprise spray-drying a plurality of microcapsules with a plurality of particulates to form a plurality of spray-dried microcapsules; wherein the microcapsules comprise a core material and a shell encapsulating the core material; wherein the spray-dried microcapsules comprise the core material and the shell encapsulating the core material; wherein the spray-dried microcapsules are coated with the particulates.

### BRIEF DESCRIPTION OF THE DRAWINGS

FIG. 1 is a schematic that illustrates an elevation view of the major components of exemplary spray drying equipment, as known in the prior art.

FIG. 2 is a flow chart that illustrates steps in a spray-drying process.

FIG. 3 illustrates an enlarged view of a liquid medium to be spray-dried, wherein the liquid medium includes a liquid, wet microcapsules, and wet particulates.

FIG. 4 illustrates a greatly enlarged view of some of the liquid medium of FIG. 3, including one of the wet microcapsules and some of the wet particulates, which have been sprayed into an atomized droplet.

FIG. 5 illustrates a greatly enlarged view of the microcapsule and particulates from FIG. 4, which have been dried.

FIG. 6 illustrates a greatly enlarged view of the dried microcapsule of FIG. 5, partially coated with the particulates of FIG. 5.

FIG. 7 illustrates an enlarged view of dried, partially coated microcapsules, including the dried microcapsule of FIG. 6, collected on a collection surface.

FIG. 8 is a micrograph showing spray dried uncoated microcapsules.

FIG. 9 is a micrograph showing spray dried partially microcapsules, resulting from a first concentration of particulates.

FIG. 10 is a micrograph showing spray dried uncoated microcapsules, resulting from a second concentration of particulates.

### DETAILED DESCRIPTION OF THE INVENTION

It has been surprisingly found that for microcapsules, a partial coating of nano-sized inorganic particulates enables such microcapsules to be successfully spray-dried in a commercially viable process. Without wishing to be bound by this theory, it is believed that this particulate coating works as described below. The particulate coating apparently helps to protect the shells from being ruptured by the hard impacts experienced by the microcapsules during the spray-drying process. The particulate coating also apparently helps to prevent the microcapsules from sticking to the inside surfaces of the drying chamber and to each other in the high heat experienced during the spray-drying process.

As a result of this particulate coating, a significant percentage of the microcapsules remain intact after spray-drying, and a significant percentage of the microcapsules can be collected from the spray drying process equipment. This allows higher process yields versus spray drying the microcapsules on their own. Further, the microcapsules are less likely to clump together during the spray-drying process when the particulates are included. This allows easier further processing for incorporation into a finished product like a composition. These benefits allow the spray-drying of microcapsules to be commercially viable.

Because the particulate coatings cover only parts of the shells for at least some of the microcapsules, the partially-coated microcapsules can release their core material in a similar way to uncoated microcapsules. The partial coatings do not fully seal up the shells. So, the coatings do not need to be opened, dissolved, or otherwise removed with an extra step. This allows the shells of the partially-coated microcapsules to be ruptured by the kind of mechanical interactions that would rupture the shells of uncoated microcapsules. The partial coatings also do not fully coat the shells of the microcapsules. So, the partial coatings do not significantly change the fracture strength profile of the outer shells or of the microcapsule. This allows the shells of the partially-coated microcapsules to be ruptured by a similar degree of force as would rupture the shells of uncoated microcapsules. As a result, the partially-coated microcapsules described herein can provide the benefits mentioned above, while still releasing their core material in a similar way to uncoated microcapsules.

While the nano-sized inorganic particulates described herein provide benefits to microcapsules like those that are friable and/or meltable, it is contemplated that such coatings can also provide benefits to various other kinds of microcapsules known in the art. It is contemplated that any of the coatings described herein can be beneficially applied to microcapsules that are friable but not necessarily meltable. Also, it is contemplated that any of the coatings described herein can be applied to microcapsules that are meltable but not necessarily friable. Further, it is contemplated that the coatings described herein may be applied to microcapsules that are neither friable nor meltable.

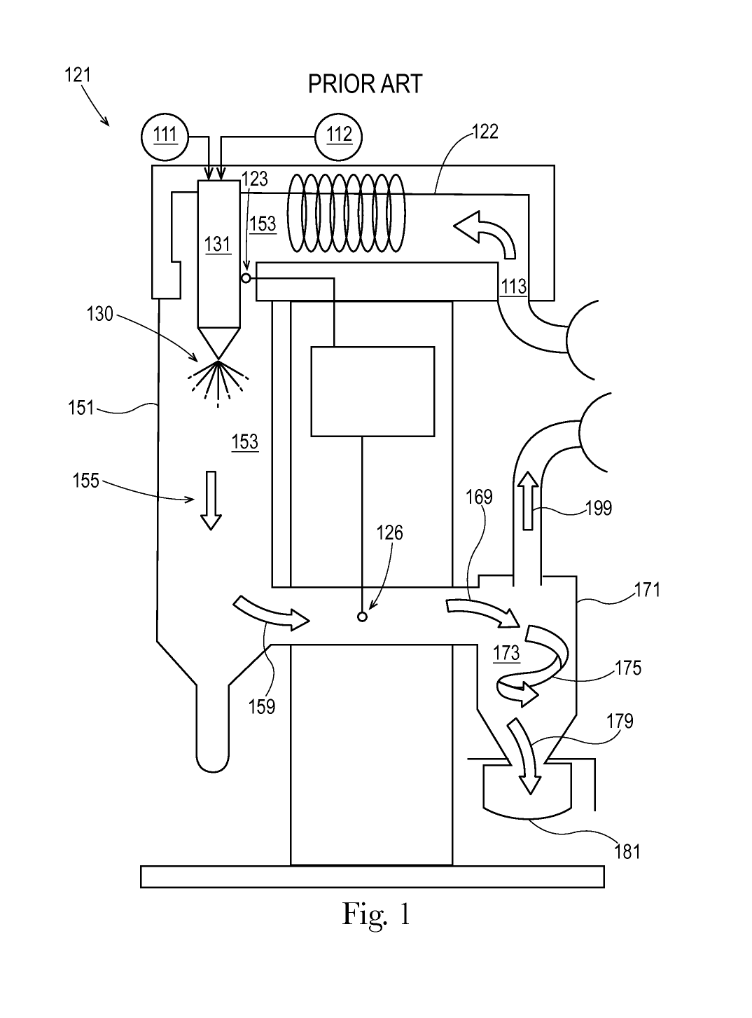

FIG. 1 is a schematic that illustrates an elevation view of major components of exemplary spray drying equipment 121, as known in the prior art.

The spray drying equipment 121 includes a heater 122, an inlet temperature sensor 123 and an outlet temperature sensor 126. The spray drying equipment 121 also includes a sprayer 131, a drying chamber 151, a cyclone chamber, 171, and a collection chamber 181. The heater 122 is optional and can be omitted. The spray drying equipment 121 can be modified to include any number of any type of additional and/or alternate spray drying equipment, configured in any way known in the art.

FIG. 1 further illustrates the materials being spray dried, and the working fluids used in the spray drying process. FIG. 1 shows a liquid medium 111 that may include one or more liquids (for example, water) and other material to be dried (e.g. microcapsules generally).

FIG. 1 also shows a pressurized gaseous working fluid 112 (for example, air) for spraying the liquid medium 111. The liquid medium 111 and the working fluid 112 are provided to the sprayer 131. The spray drying equipment 121 can use any number of any kind of working fluids known in the art. The working fluid 112 is optional and can be omitted in cases where the sprayer is a centrifugal spinning disk or wheel atomizer.

FIG. 1 shows another gaseous working fluid 113 (for example, air) for carrying and drying the wet particles. The working fluid 113 is provided to the spray drying equipment 121, and optionally heated by the heater 122 to form a heated working fluid 153. The working fluid 113 can be heated to any workable temperature known in the art. The heated working fluid 153 is transferred into the drying chamber 151. The inlet temperature sensor 123 measures the temperature of the heated working fluid 153 as it enters into the drying chamber 151. For example, the working fluid 113 can be heated, such that the temperature of the heated working fluid 153, when measured by inlet temperature sensor 123 can be 125-350 degrees Celsius, or any integer value in this range, or any range formed by any of these values for temperature.

The sprayer 131 uses the pressurized working fluid 112 to spray 130 the liquid medium 111 into the heated working fluid 153 in the drying chamber 151. Alternatively, a centrifugal atomizer may also be used to transform the liquid 111 into atomized droplets in the drying chamber. The spraying 131 forms atomized droplets that include the liquid and the microcapsules of the liquid medium 111. The heated working fluid 153 dries the liquid of the atomized droplets, leaving dried microcapsules. The heated working fluid 153 carries 155 the dried particles through drying chamber 151 and transfers 159 the dried microcapsules out of the drying chamber 151. The outlet temperature sensor 126 measures the temperature of the heated working fluid 153 as it exits the drying chamber 151. For example, the working fluid 113 can be heated, such that the temperature of the heated working fluid 153, when measured by outlet temperature sensor 126 can be 100-325 degrees Celsius, or any integer value in this range, or any range formed by any of these values for temperature.

The dried microcapsules that are transferred 159 out of the drying chamber 151 are transferred 169 into the cyclone chamber 171. The cyclone chamber 171 uses a cyclonic action 175 of a swirling gaseous working fluid 173 (for example, air) to separate the dried microcapsules out of the working fluid 173. After this separation, the working fluid 173 is transferred 199 out of the cyclone chamber 171, and the separated, dried microcapsules are transferred 179 out of the cyclone chamber 171 into the collection chamber 181. A dried microcapsule typically contains less than 10% moisture by weight.

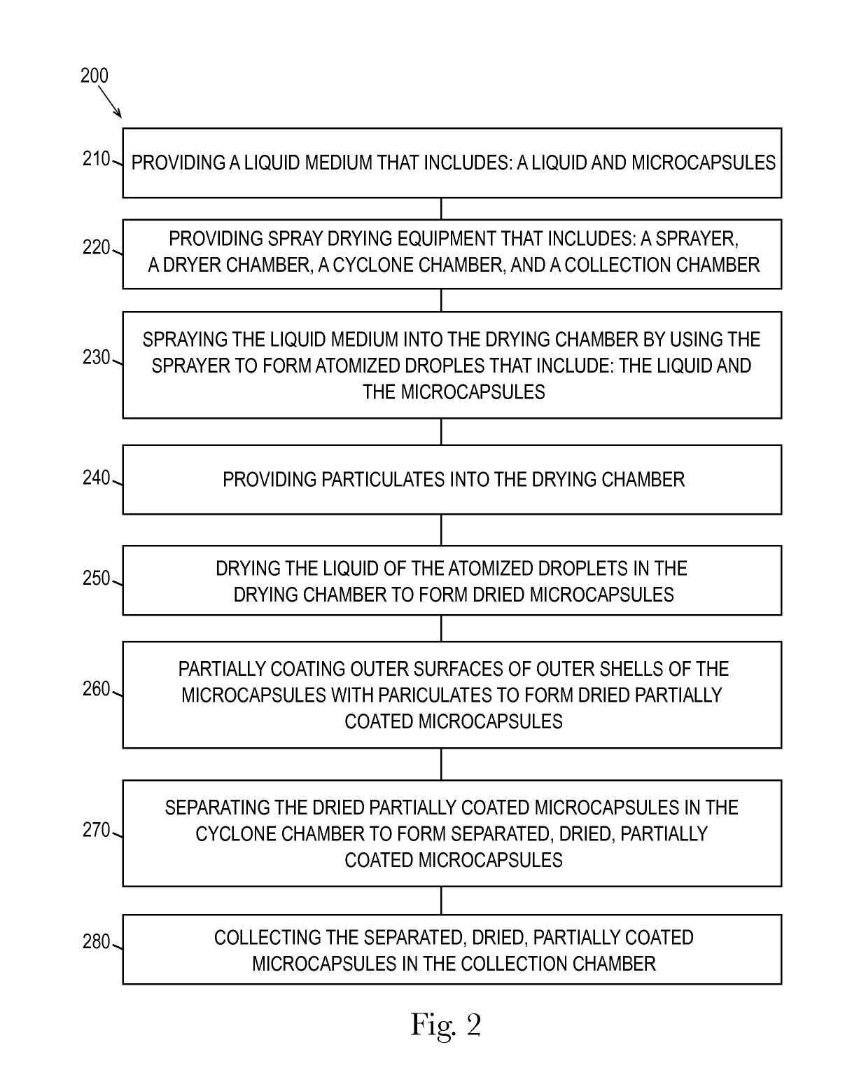

FIG. 2 is a flowchart that illustrates steps 210-280 in a spray-drying process 200. Although the steps 210-280 are described in numerical order, some or all of these steps can be performed in other orders and/or at overlapping times, and/or at the same time, as will be understood by one skilled in the art.

The spray-drying process 200 includes: a step 210 of providing a liquid medium that includes a liquid and microcapsules; a step 220 that includes providing spray drying equipment that includes: a sprayer, a drying chamber, a cyclone chamber, and a collection chamber; a step 230 that includes spraying the liquid medium into the drying chamber by using the sprayer to form atomized droplets that include the liquid and the microcapsules; a step 240 that includes providing particulates into the drying chamber; a step 250 that includes drying the liquid of the atomized droplets in the drying chamber to form dried microcapsules; a step 260 of partially coating outer surfaces of shells of the microcapsules with the particulates during the spray-drying process to form dried, partially coated microcapsules; a step 270 of separating the dried, partially coated microcapsules in the cyclone chamber, to form separated, dried, partially coated microcapsules; and a step 280 of collecting the separated, dried, partially coated microcapsules in the collection chamber.

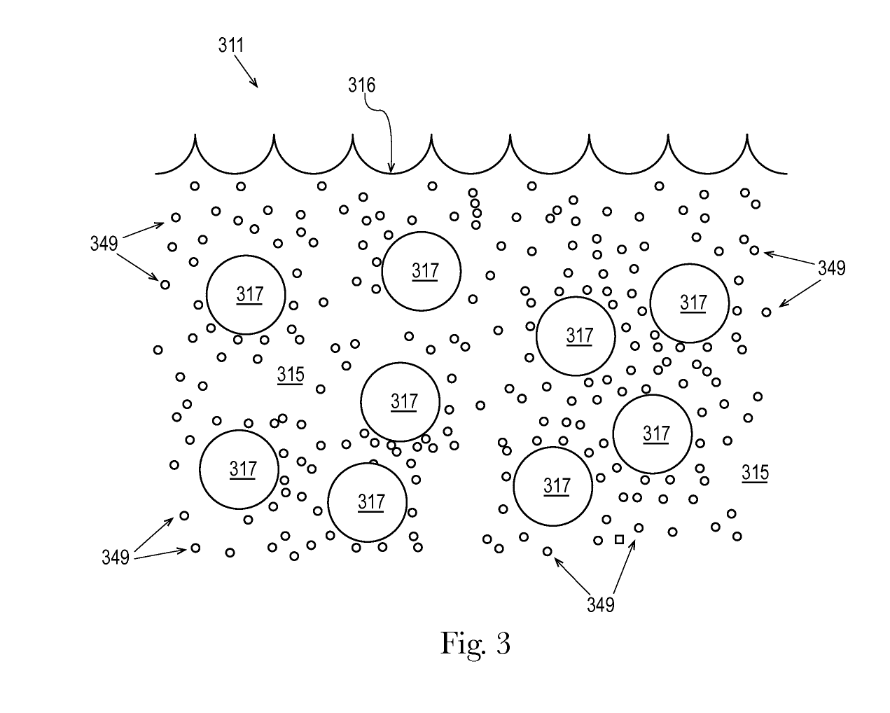

In step 210, of providing a liquid medium that includes a liquid and microcapsules, the liquid, the microcapsules, and the liquid medium can take various forms. The liquid medium can be an aqueous slurry or any other kind of liquid medium, made from one or more of any kind of liquids known in the art. For example, the liquid medium in step 210 can replace the liquid medium 111 of FIG. 1 and/or the liquid medium 311 of FIG. 3.

Some or all of the microcapsules provided in step 210 can be friable, can be meltable, can be both friable and meltable, or neither friable nor meltable. The microcapsules can have shells made from any material in any size, shape, and configuration known in the art. Some or all of the shells can include a polyacrylate material, such as a polyacrylate random copolymer. For example, the polyacrylate random copolymer can have a total polyacrylate mass, which includes ingredients selected from the group including: amine content of 0.2-2.0% of total polyacrylate mass; carboxylic acid of 0.6-6.0% of total polyacrylate mass; and a combination of amine content of 0.1-1.0% and carboxylic acid of 0.3-3.0% of total polyacrylate mass.

When a microcapsule' s shell includes a polyacrylate material, and the shell has an overall mass, the polyacrylate material can form 5-100% of the overall mass, or any integer value for percentage in this range, or any range formed by any of these values for percentage. As examples, the polyacrylate material can form at least 5%, at least 10%, at least 25%, at least 33%, at least 50%, at least 70%, or at least 90% of the overall mass.

Some or all of the shells can include one or more other materials, such as polyethylenes, polyamides, polystyrenes, polyisoprenes, polycarbonates, polyesters, polyureas, polyurethanes, polyolefins, polysaccharides, epoxy resins, vinyl polymers, and mixtures thereof.

In one aspect, useful shell materials include materials that are sufficiently impervious to the core material and the materials in the environment in which the core material is not substantially released in the environment. Suitable impervious shell materials include materials selected from the group consisting of reaction products of one or more amines with one or more aldehydes, such as urea cross-linked with formaldehyde or gluteraldehyde, melamine cross-linked with formaldehyde; gelatin-polyphosphate coacervates optionally cross-linked with gluteraldehyde; gelatin-gum Arabic coacervates; cross-linked silicone fluids; polyamine reacted with polyisocyanates; acrylate monomers polymerized via free radical polymerization, and mixtures thereof.

Some or all of the microcapsules provided in step 210 can have various fracture strengths. For at least a first group of the provided microcapsules, each microcapsule can have an outer shell with a fracture strength of 0.2-10.0 mega Pascals, when measured according to the Fracture Strength Test Method, or any incremental value expressed in 0.1 mega Pascals in this range, or any range formed by any of these values for fracture strength. As an example, a microcapsule can have an outer shell with a fracture strength of 0.2-2.0 mega Pascals.

Some or all of the microcapsules provided in step 210 can have various core to shell mass ratios. For at least a first group of the provided microcapsules, each microcapsule can have a shell, a core within the shell, and a core to shell mass ratio that is greater than or equal to: 70% to 30%, 75% to 25%, 80% to 20%, 85% to 15%, 90% to 10%, or 95% to 5%.

Some or all of the microcapsules provided in step 210 can have various shell thicknesses. For at least a first group of the provided microcapsules, some of the microcapsules can have a shell with an overall thickness of 1-300 nanometers, or any integer value for nanometers in this range, or any range formed by any of these values for thickness. As an example, microcapsules can have an shell with an overall thickness of 2-200 nanometers.

Some or all of the microcapsules provided in step 210 can have various sizes. For at least some of the microcapsules, the microcapsules can have a shell with an overall median volume-weighted particle size of 3-25 micrometers, or any integer value for micrometers in this range, or any range formed by any of these values for overall median volume-weighted particle size. Further, for at least some of the microcapsules, the overall median volume-weighted particle size of the shells can have a median value of 7-13 micrometers, or any integer value for micrometers in this range, or any range formed by any of these median values for overall median volume-weighted particle size.

Some or all of the microcapsules provided in step 210 can have various glass transition temperatures. For microcapsules encapsulating a liquid, such as a liquid fragrance, the glass transition temperature of the microcapsules and the glass transition temperature of the shell of said microcapsule are typically about the same. For at least some of the microcapsules provided, each microcapsule can have a shell with a glass transition temperature that is less than or equal to 75-150 degrees Celsius, or any integer value in this range, or any range formed by any of these values for temperature. As examples, a microcapsule can have a shell with a glass transition temperature that is less than or equal to 125 degrees Celsius, less than or equal to 105 degrees Celsius, or even less than or equal to 85 degrees Celsius.

Some or all of the microcapsules provided in step 210 can encapsulate a core material that includes one or more benefit agents. The benefit agent(s) can include one or more of chromogens, dyes, antibacterial agents, cooling sensates, warming sensates, perfumes, flavorants, sweeteners, oils, pigments, pharmaceuticals, moldicides, herbicides, fertilizers, phase change materials, adhesives, and any other kind of benefit agent known in the art, in any combination. In some examples, the perfume encapsulated can have a ClogP of less than 4.5 or a ClogP of less than 4. In some examples, the microcapsule may be anionic, cationic, zwitterionic, or have a neutral charge.

In some examples, the microcapsule's shell comprises a reaction product of a first mixture in the presence of a second mixture comprising an emulsifier, the first mixture comprising a reaction product of i) an oil soluble or dispersible amine with ii) a multifunctional acrylate or methacrylate monomer or oligomer, an oil soluble acid and an initiator, the emulsifier comprising a water soluble or water dispersible acrylic acid alkyl acid copolymer, an alkali or alkali salt, and optionally a water phase initiator. In some examples, said amine is an aminoalkyl acrylate or aminoalkyl methacrylate.

In some examples, the microcapsules include a core material and a shell surrounding the core material, wherein the shell comprises: a plurality of amine monomers selected from the group consisting of aminoalkyl acrylates, alkyl aminoalkyl acrylates, dialkyl aminoalykl acrylates, aminoalkyl methacrylates, alkylamino aminoalkyl methacrylates, dialkyl aminoalykl methacrylates, tertiarybutyl aminethyl methacrylates, diethylaminoethyl methacrylates, dimethylaminoethyl methacrylates, dipropylaminoethyl methacrylates, and mixtures thereof; and a plurality of multifunctional monomers or multifunctional oligomers.

The liquid medium of 210 can include any workable amount of the microcapsules disclosed herein, and may also include any workable amount of one or more of any other microcapsule known in the art.

Step 210 may be eliminated, and step 240 of spraying can be performed by providing microcapsules to the sprayer in any other way known in the art.

In step 220, of providing spray drying equipment, the sprayer can be the sprayer 131 of FIG. 1, the drying chamber can be the drying chamber 151 of FIG. 1, the cyclone chamber can be the cyclone chamber 171 of FIG. 1, and the collection chamber can be the collection chamber 181 of FIG. 1, configured accordingly as disclosed herein or known in the art.

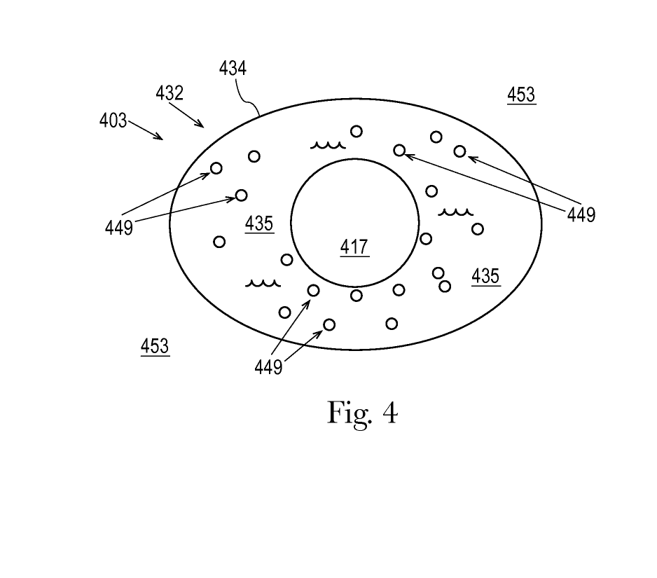

In step 230, of spraying the liquid medium into the drying chamber by using the sprayer, to form atomized droplets that include the liquid and the microcapsules, the atomized droplets can take various forms, including any form disclosed herein or known in the art. For example, some or all of the atomized droplets in step 230 can have the form of the atomized droplet 432 of FIG. 4.

In step 240, of providing particulates into the drying chamber, the providing can be accomplished in various ways and the particulates can take various forms, including any form disclosed herein or known in the art.

Some or all of the particulates provided in step 240 can be inorganic particulates, such as silica particulates, including silica particulates made of silicon dioxide. For example, the silica particulates can be precipitated silicas, colloidal silicas, fumed silicas, and/or other kinds of silicas known in the art, and/or mixtures thereof. Alternatively, some or all of the inorganic particulates can include particulates made from one or more of citric acid, sodium carbonate, sodium sulfate, magnesium chloride, potassium chloride, sodium chloride, sodium silicate, modified cellulose, zeolite and any other kind of inorganic particulate known in the art, in any combination.

Some or all of the particulates provided in step 240 can have various sizes. For at least a first group of the provided particulates, the particulates can have an overall median volume-weighted particle size of 1-999 nanometers, or any integer value for nanometers in this range, or any range formed by any of these values for overall median volume-weighted particle size. As an example, the particulates can have an overall thickness of 1-50 nanometers or from 5-50 nanometers

Some or all of the particulates provided in step 240 can be provided in various forms. As an example, the particulates can be provided in a liquid medium such as a solution or a colloidal suspension.

The particulates provided in step 240 can be provided in various ways. The particulates can be provided into the drying chamber as wet particulates by including them in the liquid medium of the first step 210, which is sprayed in the second step 220. FIG. 3 illustrates wherein the liquid medium 311 to be spray-dried, includes a liquid 315, microcapsules 317, and particulates 349. Step 240 can be completed as part of step 210 and step 220. As an example, silica particulates can be provided in a colloidal suspension that is added to an aqueous slurry that includes microcapsules, to create an aqueous slurry that includes the microcapsules and the silica particulates, and that aqueous slurry can then be sprayed.

The particulates can be provided into the drying chamber as wet particulates by including them in another liquid medium, separate from the liquid medium of the first step 210, wherein the other liquid medium is sprayed into the drying chamber separate from the spraying in the second step 220. Alternatively, the particulates can be added to the drying chamber any other way known in the art. For example, it is contemplated that it may be possible to provide the particulates to the drying chamber as dry particulates.

The particulates provided in step 240 can be provided in any workable amount of any of the particulates disclosed herein, and may also include any workable amount of one or more of any other particulates known in the art.

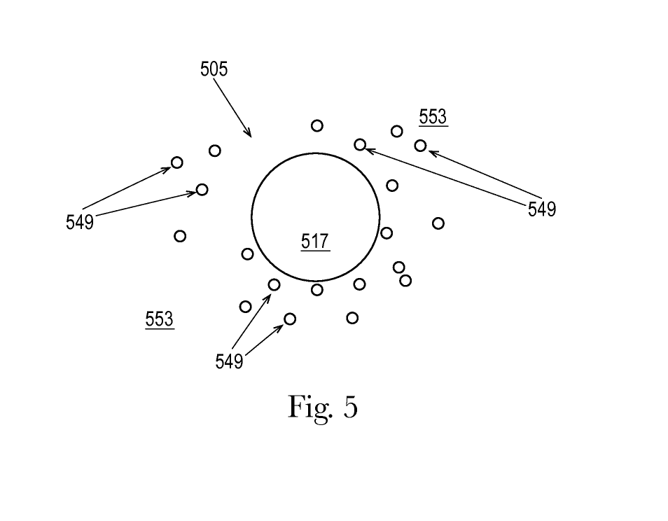

In step 250, of drying the liquid of the atomized droplets in the drying chamber, to form dried microcapsules, the dried microcapsules can take various forms, including any form disclosed herein or known in the art. For example, some or all of the dried microcapsules in the fifth step 250 can have the form of the dried microcapsule 517 of FIG. 5.

The drying can include drying the microcapsules by using a working fluid that is heated to a temperature that is greater than the glass transition temperature of the microcapsules. For example, the drying can include drying the microcapsules by using a working fluid heated to an average temperature that is 25-175 degrees Celsius greater than the glass transition temperature of the microcapsules. As another example, the drying can includes drying the microcapsules by using a working fluid heated to an average temperature that is 50-100 degrees Celsius greater than the glass transition temperature of the microcapsules. The higher temperature of the working fluid with respect to the glass transition temperature of the microcapsules helps to prevent premature fracturing during the spray-drying process.

In step 260, the outer surfaces of the shells of the dried microcapsules from step 250 can be partially coated, to form spray-dried microcapsules that are coated with particulates. For example, the coating can include partially coating the spray-dried microcapsules, such that, for at least a first group of the spray-dried microcapsules, 15-85% of an outer surface of the shell of each microcapsule is coated by the particulates. As another example, the coating can include only partially coating the spray-dried microcapsules, such that, for at least a first group of the spray-dried microcapsules, 30-70% of an outer surface of the shell of the microcapsules are coated by the particulates.

In step 270, the spray-dried microcapsules from step 260 can be separated in a cyclone chamber, such as the cyclone chamber 171 of FIG. 1, to form separated, spray-dried microcapsules.

In step 280, the separated, spray-dried microcapsules from step 270 can be collected in a collection chamber, such as the collection chamber 181 of FIG. 1. As a result of the particulate coating described above, a significant percentage of the spray-dried microcapsules remain intact after spray-drying such that the spray-dried microcapsules include the core material and the shell encapsulating the core material. Also, the process allows for a significant percentage of the spray-dried microcapsules to be collected from the spray drying process equipment. This produces high process yields, which allows the spray-drying process 200 to be commercially viable for microcapsules, including but not limited to, friable and/or meltable microcapsules.

The spray-drying process 200 can be used to produce a process yield of 60-95% of intact, spray-dried microcapsules, or any integer value for percentage in this range, or any range formed by any of these values for percentage, when measured according to the Process Yield Test Method. As examples, the spray-drying process can be used to produce a process yield of 70-95% of intact, spray-dried microcapsules or a process yield of 80-95% of intact, spray-dried microcapsules or a process yield of 90-95% of intact, spray-dried microcapsules. The process may also yield greater than 22% but less than or equal to 66% of the intact, spray-dried microcapsules according to the Process Yield Test Method. The process may also yield greater than 22% but less than or equal to 95%.

FIG. 3 illustrates an enlarged view of a liquid medium 311 to be spray-dried, wherein the liquid medium 311 includes a liquid 315, a liquid surface 316, microcapsules 317, and particulates 349. The liquid medium 311 is an aqueous slurry, which can be configured in any way disclosed herein or known in the art. The liquid medium 311 can also take various other forms, including any form disclosed herein or known in the art.

The microcapsules 317 are suspended in the liquid medium 311. The microcapsules 317 can be configured in any way disclosed herein or known in the art. Some or all of the microcapsules 317 can also take various other forms, including any form disclosed herein or known in the art.

The particulates 349 are silica particulates, which can be configured in any way disclosed herein or known in the art. Some or all of the particulates 349 can also take various other forms, including any form disclosed herein or known in the art. The particulates 349 may be a soluble species, that upon drying, causes precipitation of these dissolved species onto the microcapsule surface.

The liquid medium 311 can be spray-dried according to the method 200 of FIG. 2. Specifically, the liquid medium 311 can be sprayed into a drying chamber by using a sprayer, according to step 230 of the method 200 of FIG. 2. The liquid medium 311 may not include the particulates 317; the particulates may be provided wet, dry, or in some other way.

FIG. 4 illustrates a greatly enlarged view of part 403 of an inside of a drying chamber, into which the liquid medium 311 of FIG. 3 has been sprayed. FIG. 4 shows an atomized droplet 432 being carried and dried by a heated working fluid 453. The droplet 432 is formed from some of the liquid medium 311 of FIG. 3, which has been sprayed by using a sprayer, according to step 230 of the method 200 of FIG. 2.

The droplet 432 includes microcapsule 417, particulates 449, and sprayed liquid medium 435. The microcapsule 417 is one of the microcapsules 317 of FIG. 3. The particulates 449 are some of the particulates 349 of FIG. 3. The liquid medium 435 is some of the liquid medium 311 of FIG. 3. The microcapsule 417 and the particulates 449 are suspended in the liquid medium 435. The droplet 432 includes an outer wall 434.

The droplet 432 can be carried through and dried in the drying chamber, according to step 250 of the method 200 of FIG. 2. FIG. 4 is intended to show the components found in the droplet 432, and to indicate their relative differences in size. However, spray-dried droplets can have various sizes and shapes, and can include various numbers of microcapsules and particulates.

FIG. 5 illustrates a greatly enlarged view of part 505 of an inside of a drying chamber, into which the liquid medium 311 of FIG. 3 has been sprayed. FIG. 5 illustrates a greatly enlarged view 553 of the microcapsule 517 and particulates 549 from FIG. 4.

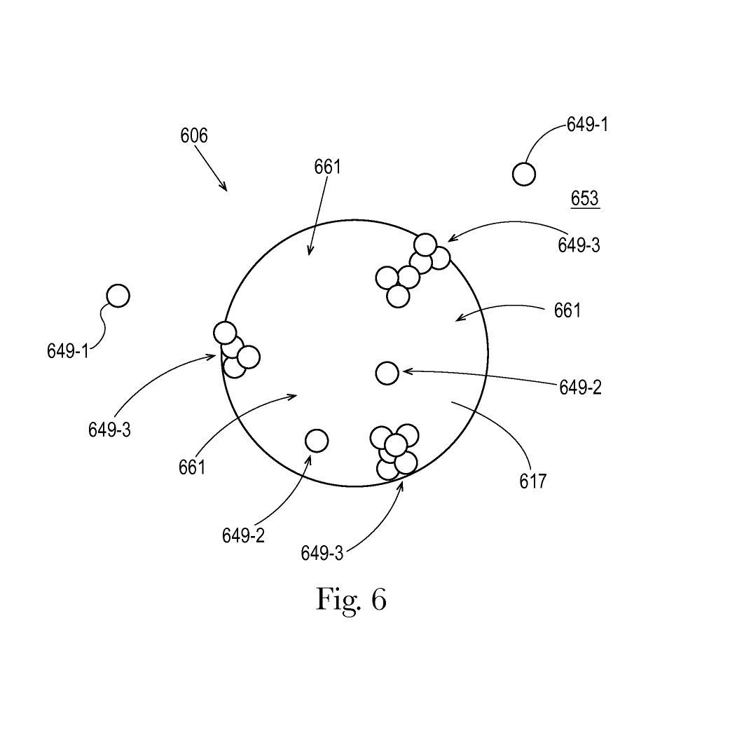

FIG. 6 illustrates a greatly enlarged view 653 of a spray-dried microcapsule 617, which is the microcapsule 517 of FIG. 5, partially coated with the particulates 549 of FIG. 5. The spray-dried microcapsule 617 is an example of one that may be present in the collection chamber 606 after spray drying. Note the presence of the shell 661 of the spray-dried microcapsule 617. Also, note that the shell 661 of the spray-dried microcapsule 617 may be coated with a unitary particulate 649-2 and clumps of particulates 649-3, and that the shell 661 of the spray-dried microcapsule 617 is only partially coated with the unitary particulate 649-2 and the clumps of particulates 649-3. Also potentially present in the collection chamber 606 may be free particulates 649-1 that have not coated the shell 661 of the spray-dried microcapsule 617.

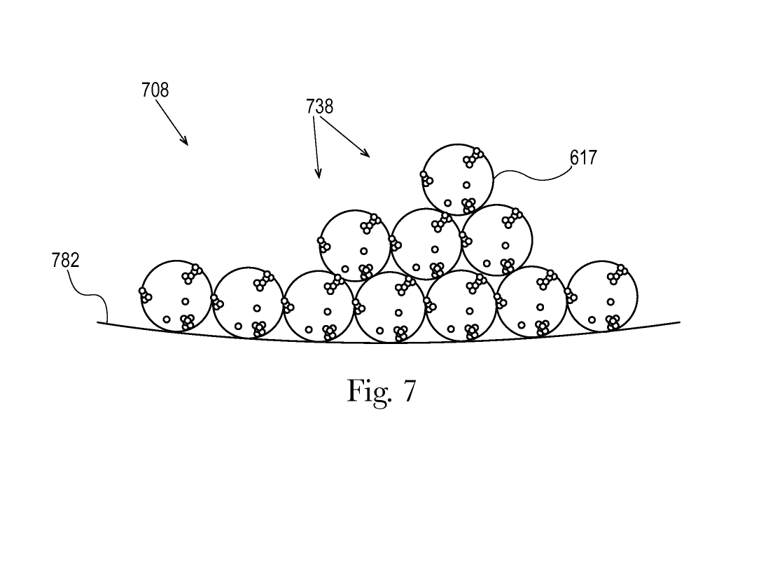

FIG. 7 illustrates an enlarged view 708 of spray-dried, partially coated microcapsules 738, including the spray-dried microcapsule 617 of FIG. 6, collected on a collection surface 782. The collected spray-dried microcapsules can have a bulk flow energy of 1-800 millijoules, of 1-500 millijoules, or of 1-200 millijoules, when tested according to the Bulk Flow Energy Test Method.

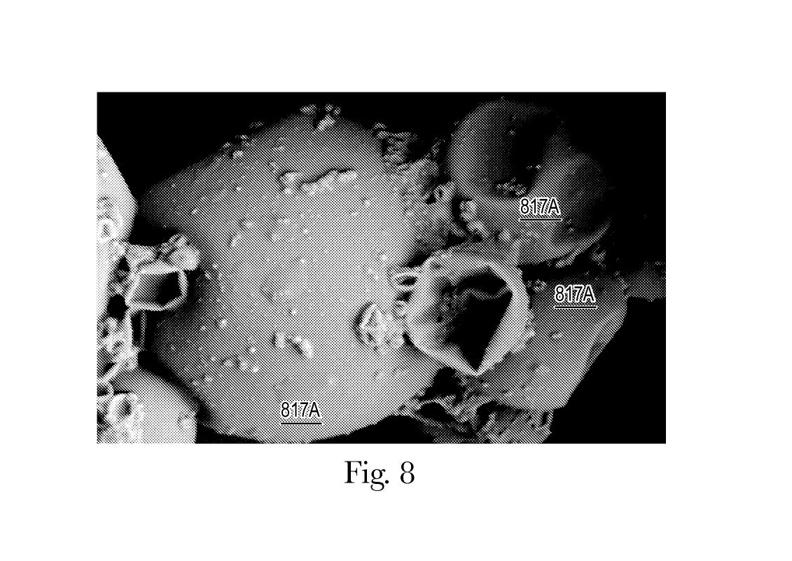

FIG. 8 is a micrograph showing spray-dried, uncoated microcapsules 817A.

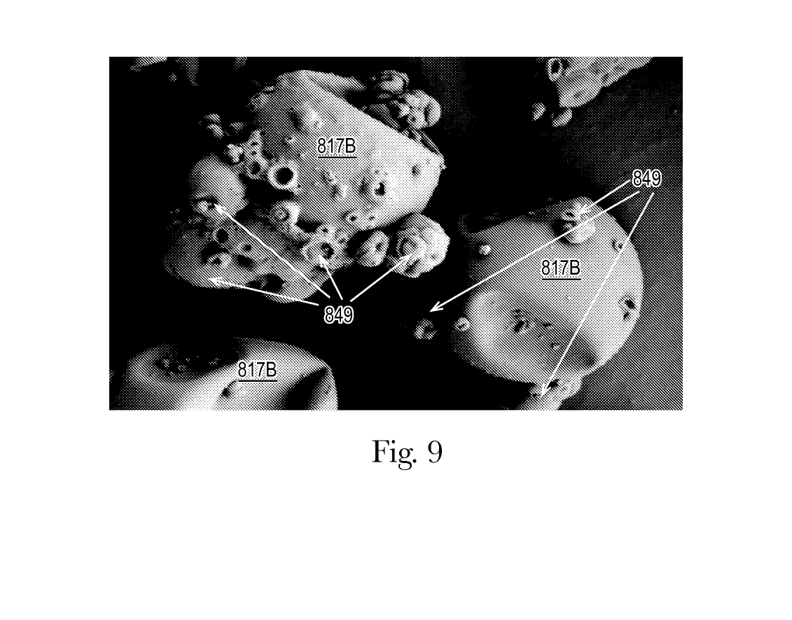

FIG. 9 is a micrograph showing spray-dried microcapsules 817B partially coated with particulates 849, from a 1.5% colloidal silica (Ludox HS-30) process aid in the slurry, as described herein.

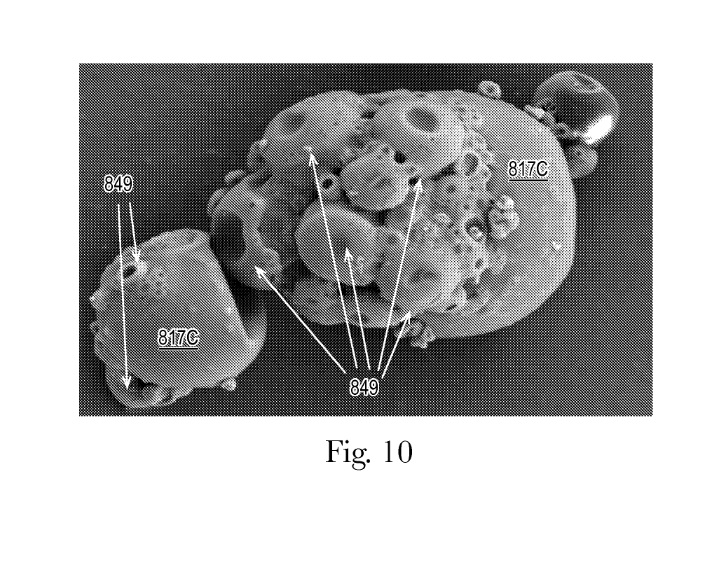

FIG. 10 is a micrograph showing spray-dried microcapsules 817C partially coated with particulates 849, from a 3% colloidal silica (Ludox HS-30) process aid in the slurry, as described herein.

Various (hydrous or anhydrous) compositions can comprise the microcapsules produced by the spray-drying process 200 of FIG. 2, including: a fluid fabric enhancer; a solid fabric enhancer; a fluid shampoo; a solid shampoo; a powder shampoo; a powder hair or skin refresher; a fluid skin care formulation; a solid skin care formulation; hair conditioner; body wash, body spray, bar soap, hand sanitizer, solid antiperspirant, semi-solid antiperspirant, fluid antiperspirant, solid deodorant, semi-solid deodorant, fluid deodorant, fluid detergent, solid detergent, fluid hard surface cleaner, solid hard surface cleaner; and a unit dose detergent comprising a detergent and a water soluble film encapsulating said detergent.

The non-limiting list of adjunct ingredients illustrated hereinafter are suitable for use in compositions and may be desirably incorporated, for example, to assist or enhance performance, for treatment of the substrate to be cleaned, or to modify the aesthetics of the composition as is the case with perfumes, colorants, dyes or the like. It is understood that such adjuncts are in addition to the components that are supplied via the spray-dried microcapsules. The precise nature of these adjunct ingredients, and levels of incorporation thereof, will depend on the physical form of the composition and the nature of the operation for which it is to be used. Suitable adjunct materials include, but are not limited to, polymers, for example cationic polymers, surfactants, builders, chelating agents, dye transfer inhibiting agents, dispersants, enzymes, enzyme stabilizers, catalytic materials, bleach activators, polymeric dispersing agents, clay soil removal/anti-redeposition agents, brighteners, suds suppressors, dyes, additional perfume and perfume delivery systems, structure elasticizing agents, fabric softeners, carriers, hydrotropes, processing aids and/or pigments, antiperspirant actives, skin care actives (e.g. nicacinamide), glycerin, and mixtures thereof. In some examples, the adjunct may be a carrier like water. It is also envisioned that more than one type of adjunct ingredient may be included in the composition.

The compositions may be used as consumer products (i.e. products intended to be sold to consumers without further modification or processing). Moreover, the spray-dried microcapsules may be applied to any article, such as a fabric or any absorbent material including, but not limited to, feminine hygiene products, diapers, and adult incontinence products. The composition may also be incorporated into an article.

### Solid Antiperspirant Compositions

Anhydrous compositions, like solid antiperspirant compositions, may require microcapsules with less than 20% water, preferably with less than 5% water. Free water in such anhydrous compositions can lead to the crystallization of the antiperspirant actives which may affect the performance of the composition when used. Spray-drying a slurry of microcapsules before inclusion into a solid antiperspirant composition is one way of reducing the amount of water associated with the microcapsules. However, it has been found that the conventional process for spray-drying may lead to poor yields of spray-dried microcapsules. Such poor yields cannot often be around 20%. It has been surprisingly discovered that when microcapsules are spray-dried with particulates, like those described herein, said particulates improve the process yield without significantly compromising the microcapsules' performance benefit. Thus, the process of spray-drying microcapsules with particulates may be beneficial for producing solid antiperspirant compositions that include microcapsules.

Additionally, for at least some friable microcapsules, such microcapsules may be more flexible in environments containing high levels of water. For example, for at least some microcapsules, said microcapsules may not release their core material (e.g. a fragrance) when friction or other mechanical forces are applied in a hyper-hydrated state. By spray-drying said microcapsules before inclusion in the composition, said microcapsules may be more likely to rupture and release their core materials.

Solid antiperspirant compositions may include an antiperspirant active suitable for application to human skin. The concentration of the antiperspirant active in the composition should be sufficient to provide the desired enhanced wetness protection. For example, the active may be present in an amount of from about 0.1%, about 0.5%, about 1%, about 5%, or about 10%; to about 60%, about 35%, about 25% or about 20%, by weight of the composition. These weight percentages are calculated on an anhydrous metal salt basis exclusive of water and any complexing agents such as glycine, glycine salts, or other complexing agents.

An antiperspirant active can include any compound, composition, or other material having antiperspirant activity. Such actives may include astringent metallic salts, especially inorganic and organic salts of aluminum, zirconium and zinc, as well as mixtures thereof. For example, the antiperspirant actives may include zirconium-containing salts or materials, such as zirconyl oxyhalides, zirconyl hydroxyhalides, and mixtures thereof; and/or aluminum-containing salts such as, for example, aluminum halides, aluminum chlorohydrate, aluminum hydroxyhalides, and mixtures thereof.

1. Aluminum Salts

Aluminum salts useful herein can include those that conform to the formula:

Al₂(OH)ₐCl_{b}.xH₂O

wherein a is from about 2 to about 5; the sum of a and b is about 6; x is from about 1 to about 6; where a, b, and x may have non-integer values. For example, aluminum chlorohydroxides referred to as “5/6 basic chlorohydroxide,” wherein a is about 5 and “2/3 basic chlorohydroxide”, wherein a=4 may be used.

2. Zirconium Salts

Zirconium salts useful herein can include those which conform to the formula:

ZrO(OH)₂₋ₐClₐ.xH₂O

wherein a is from about 1.5 to about 1.87; x is from about 1 to about 7; and wherein a and x may both have non-integer values. Useful are zirconium salt complexes that additionally contain aluminum and glycine, commonly known as “ZAG complexes”. These complexes can contain aluminum chlorohydroxide and zirconyl hydroxy chloride conforming to the above-described formulas. Examples of two such complexes include aluminum zirconium trichlorohydrex and aluminum zirconium tetrachlorohydrex.

Antiperspirant compositions can also include a structurant to help provide the composition with the desired viscosity, rheology, texture and/or product hardness, or to otherwise help suspend any dispersed solids or liquids within the composition. The term “structurant” may include any material known or otherwise effective in providing suspending, gelling, viscosifying, solidifying, or thickening properties to the composition or which otherwise provide structure to the final product form. These structurants may include, for example, gelling agents, polymeric or nonpolymeric agents, inorganic thickening agents, or viscosifying agents. The thickening agents may include, for example, organic solids, silicone solids, crystalline or other gellants, inorganic particulates such as clays or silicas, or combinations thereof.

The concentration and type of the structurant selected for use in the antiperspirant composition will vary depending upon the desired product form, viscosity, and hardness. The thickening agents suitable for use herein, may have a concentration range from about 0.1%, about 2%, about 3%, about 5%; or about 10%; to about 35%, about 20%, about 10%, or about 8%, by weight of the composition. Soft solids will often contain a lower amount of structurant than solid compositions. For example, a soft solid may contain from about 1.0% to about 9%, by weight of the composition, while a solid composition may contain from about 15% to about 25%, by weight of the composition, of structurant. This is not a hard and fast rule, however, as a soft solid product with a higher structurant value can be formed by, for example, shearing the product as it is dispensed from a package.

Non-limiting examples of suitable gelling agents include fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, triglycerides, sucrose esters such as SEFA behenate, inorganic materials such as clays or silicas, other amide or polyamide gellants, and mixtures thereof.

Suitable gelling agents include fatty acid gellants such as fatty acid and hydroxyl or alpha hydroxyl fatty acids, having from about 10 to about 40 carbon atoms, and ester and amides of such gelling agents. Non-limiting examples of such gelling agents include, but are not limited to, 12-hydroxystearic acid, 12-hydroxylauric acid, 16-hydroxyhexadecanoic acid, behenic acid, eurcic acid, stearic acid, caprylic acid, lauric acid, isostearic acid, and combinations thereof. Preferred gelling agents are 12-hydroxystearic acid, esters of 12-hydroxystearic acid, amides of 12-hydroxystearic acid and combinations thereof.

Other suitable gelling agents include amide gellants such as di-substituted or branched monoamide gellants, monsubstituted or branched diamide gellants, triamide gellants, and combinations thereof, including n-acyl amino acid derivatives such as n-acyl amino acid amides, n-acyl amino acid esters prepared from glutamic acid, lysine, glutamine, aspartic acid, and combinations thereof.

Still other examples of suitable gelling agents include fatty alcohols having at least about 8 carbon atoms, at least about 12 carbon atoms but no more than about 40 carbon atoms, no more than about 30 carbon atoms, or no more than about 18 carbon atoms. For example, fatty alcohols include but are not limited to cetyl alcohol, myristyl alcohol, stearyl alcohol and combinations thereof.

Non-limiting examples of suitable tryiglyceride gellants include tristearin, hydrogenated vegetable oil, trihydroxysterin (Thixcin® R, available from Rheox, Inc.), rape seed oil, castor wax, fish oils, tripalmitin, Syncrowax® HRC and Syncrowax® HGL-C (Syncrowax® available from Croda, Inc.).

Other suitable thickening agents include waxes or wax-like materials having a melt point of above 65° C., more typically from about 65° C. to about 130° C., examples of which include, but are not limited to, waxes such as beeswax, carnauba, bayberry, candelilla, montan, ozokerite, ceresin, hydrogenated castor oil (castor wax), synthetic waxes and microcrystalline waxes. Castor wax is preferred within this group. The synthetic wax may be, for example, a polyethylene, a polymethylene, or a combination thereof. Some suitable polymethylenes may have a melting point from about 65° C. to about 75° C. Examples of suitable polyethylenes include those with a melting point from about 60° C. to about 95° C.

Further structurants for use in the solid antiperspirant compositions of the present invention may include inorganic particulate thickening agents such as clays and colloidal pyrogenic silica pigments. For example, colloidal pyrogenic silica pigments such as Cab-O-Sil®, a submicroscopic particulated pyrogenic silica may be used. Other known or otherwise effective inorganic particulate thickening agents that are commonly used in the art can also be used in the solid antiperspirant compositions of the present invention. Concentrations of particulate thickening agents may range, for example, from about 0.1%, about 1%, or about 5%; to about 35%, about 15%, about 10% or about 8%, by weight of the composition.

Suitable clay structurants include montmorillonite clays, examples of which include bentonites, hectorites, and colloidal magnesium aluminum silicates. These and other suitable clays may be hydrophobically treated, and when so treated will generally be used in combination with a clay activator. Non-limiting examples of suitable clay activators include propylene carbonate, ethanol, and combinations thereof. When clay activators are present, the amount of clay activator will typically range from about 40%, about 25%, or about 15%; to about 75%, about 60%, or about 50%, by weight of the clay.

Solid antiperspirant compositions may further include anhydrous liquid carriers. These are present, for example, at concentrations ranging from about 10%, about 15%, about 20%, about 25%; to about 99%, about 70%, about 60%, or about 50%, by weight of the composition. Such concentrations will vary depending upon variables such as product form, desired product hardness, and selection of other ingredients in the composition. The anhydrous carrier may be any anhydrous carrier known for use in personal care applications or otherwise suitable for topical application to the skin For example, anhydrous carriers may include, but are not limited to volatile and nonvolatile fluids.

An antiperspirant composition may further include a volatile fluid such as a volatile silicone carrier. Volatile fluids are present, for example, at concentrations ranging from about 20% or from about 30%; to about 80%, or no about 60%, by weight of the composition. The volatile silicone of the solvent may be cyclic, linear, and/or branched chain silicone. “Volatile silicone”, as used herein, refers to those silicone materials that have measurable vapor pressure under ambient conditions.

The volatile silicone may be a cyclic silicone. The cyclic silicone may have from about 3 silicone atoms, or from about 5 silicone atoms; to about 7 silicone atoms, or about 6 silicone atoms. For example, volatile silicones may be used which conform to the formula:

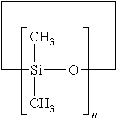

wherein n is from about 3, or from about 5; to about 7, or about 6. These volatile cyclic silicones generally have a viscosity of less than about 10 centistokes at 25° C. Suitable volatile silicones for use herein include, but are not limited to, Cyclomethicone D5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.). SWS-03314, SWS-03400, F-222, F-223, F-250, F-251 (available from SWS Silicones Corp.); Volatile Silicones 7158, 7207, 7349 (available from Union Carbide); Masil SF-V (available from Mazer) and combinations thereof.

An antiperspirant composition may further comprise a non-volatile fluid. These non-volatile fluids may be either non-volatile organic fluids or non-volatile silicone fluids. The non-volatile organic fluid can be present, for example, at concentrations ranging from about 1%, from about 2%; to about 20%, or about 15%, by weight of the composition.

Non-limiting examples of nonvolatile organic fluids include, but are not limited to, mineral oil, PPG-14 butyl ether, isopropyl myristate, petrolatum, butyl stearate, cetyl octanoate, butyl myristate, myristyl myristate, C12-15 alkylbenzoate (e.g., Finsolv™), dipropylene glycol dibenzoate, PPG-15 stearyl ether benzoate and blends thereof (e.g. Finsolv TPP), neopentyl glycol diheptanoate (e.g. Lexfeel 7 supplied by Inolex), octyldodecanol, isostearyl isostearate, octododecyl benzoate, isostearyl lactate, isostearyl palmitate, isononyl/isononoate, isoeicosane, octyldodecyl neopentanate, hydrogenated polyisobutane, and isobutyl stearate.

An antiperspirant composition may further include a non-volatile silicone fluid. The non-volatile silicone fluid may be a liquid at or below human skin temperature, or otherwise in liquid form within the anhydrous antiperspirant composition during or shortly after topical application. The concentration of the non-volatile silicone may be from about 1%, from about 2%; to about 15%, about 10%, by weight of the composition. Nonvolatile silicone fluids of the present invention may include those which conform to the formula:

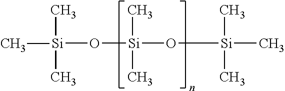

wherein n is greater than or equal to 1. These linear silicone materials may generally have viscosity values of from about 5 centistokes, from about 10 centistokes; to about 100,000 centistokes, about 500 centistokes, about 200 centistokes, or about 50 centistokes, as measured under ambient conditions.

Specific non limiting examples of suitable nonvolatile silicone fluids include Dow Corning 200, hexamethyldisiloxane, Dow Corning 225, Dow Corning 1732, Dow Corning 5732, Dow Corning 5750 (available from Dow Corning Corp.); and SF-96, SF-1066 and SF18(350) Silicone Fluids (available from G.E. Silicones).

Low surface tension non-volatile solvent may be also be used. Such solvents may be selected from the group consisting of dimethicones, dimethicone copolyols, phenyl trimethicones, alkyl dimethicones, alkyl methicones, and mixtures thereof. Low surface tension non-volatile solvents are also described in U.S. Pat. No. 6,835,373 (Kolodzik et al.).

An antiperspirant composition may include a malodor reducing agent. Malodor reducing agents include components other than the antiperspirant active within the composition that act to eliminate the effect that body odor has on fragrance display. These agents may combine with the offensive body odor so that they are not detectable including, but not limited to, suppressing evaporation of malodor from the body, absorbing sweat or malodor, masking the malodor or microbiological activity on odor causing organisms. The concentration of the malodor reducing agent within the composition is sufficient to provide such chemical or biological means for reducing or eliminating body odor. Although the concentration will vary depending on the agent used, generally, the malodor reducing agent may be included within the composition from about 0.05%, about 0.5%, or about 1%; to about 15%, about 10%, or about 6%, by weight of the composition.

Malodor reducing agents may include, but are not limited to, pantothenic acid and its derivatives, petrolatum, menthyl acetate, uncomplexed cyclodextrins and derivatives thereof, talc, silica and mixtures thereof.

For example, if panthenyl triacetate is used, the concentration of the malodor reducing agent may be from about 0.1% or about 0.25%; to about 3.0%, or about 2.0%, by weight of the composition. Another example of a malodor reducing agent is petrolatum which may be included from about 0.10%, or about 0.5%; to about 15%, or about 10%, by weight of the composition. A combination may also be used as the malodor reducing agent including, but not limited to, panthenyl triacetate and petrolatum at levels from about 0.1%, or 0.5%; to about 3.0%, or about 10%, by weight of the composition. Menthyl acetate, a derivative of menthol that does not have a cooling effect, may be included from about 0.05%, or 0.01%; to about 2.0%, or about 1.0%, by weight of the composition. The malodor reducing agent may be in the form of a liquid or a semi-solid such that it does not contribute to product residue.

### Test Methods

### Test Method for Determining Median Volume-Weighted Particle Size of Microcapsules

One skilled in the art will recognize that various protocols may be constructed for the extraction and isolation of microcapsules from finished products, and will recognize that such methods require validation via a comparison of the resulting measured values, as measured before and after the microcapsules' addition to and extraction from the finished product. The isolated microcapsules are then formulated in deionized water to form a capsule slurry for characterization for particle size distribution.

The median volume-weighted particle size of the microcapsules is measured using an Accusizer 780A, made by Particle Sizing Systems, Santa Barbara Calif., or equivalent. The instrument is calibrated from 0 to 300 μm using particle size standards (as available from Duke/Thermo-Fisher-Scientific Inc., Waltham, Mass., USA). Samples for particle size evaluation are prepared by diluting about 1 g of capsule slurry in about 5 g of de-ionized water and further diluting about 1 g of this solution in about 25 g of water. About 1 g of the most dilute sample is added to the Accusizer and the testing initiated using the autodilution feature. The Accusizer should be reading in excess of 9200 counts/second. If the counts are less than 9200 additional sample should be added. Dilute the test sample until 9200 counts/second and then the evaluation should be initiated. After 2 minutes of testing the Accusizer will display the results, including the median volume-weighted particle size.

### Test Method for Determining Percent Coating of a Surface of a Shell

One skilled in the art will recognize that various protocols may be constructed for the extraction and isolation of microcapsules from finished products, and will recognize that such methods require validation via a comparison of the resulting measured values, as measured before and after the microcapsules' addition to and extraction from the finished product. The isolated microcapsules are then formulated in DI water to form a slurry for characterization.

TA Instruments, TGA Q5000, or equivalent is used to perform the thermal gravimetric analysis. All samples (i.e. capsule slurries) are placed in hermetically sealed, aluminum punch pans. Samples are heated under nitrogen atmosphere flowing at 25 ml/min. using the step thermal profile described in Table 1.

TABLE 1
TGA Analysis Ramp Profile

| | | | Final | |
|---|---|---|---|---|
| | | Rate | Temperature | Time |
| Step | Isothermal/Ramp | (C. °/min) | (C. °) | (Minutes) |
| 1 | Isothermal | | 25-45 | 30 |
| 2 | Ramp | 5 | 65 | 4-8 |
| 3 | Isothermal | | 65 | 30 |
| 4 | Ramp | 10 | 85 | 2 |
| 5 | Isothermal | | 85 | 30 |
| 6 | Ramp | 10 | 120 | 3.5 |
| 7 | Isothermal | | 120 | 30 |
| 8 | Ramp | 10 | 200 | 8 |
| 9 | Isothermal | | 200 | 30 |
| 10 | Ramp | 10 | 250 | 5 |
| 11 | Isothermal | | 250 | 15 |
| 12 | Ramp | 10 | 350 | 5 |
| 13 | Isothermal | | 350 | 15 |
| 14 | Ramp | 10 | 450 | 5 |
| 15 | Isothermal | | 450 | 15 |
| Total Approximate | | | | 230 |
| Analysis Time | | | | |

Note that in the following TGA graph, the percent mass loss is plotted on the left, primary Y axis against time on the X axis. The temperature is plotted on the right, secondary Y axis.
Note there was less than 1% mass loss by the time the instrument reached 65° C. Mass loss thereafter was considered as either volatile perfume mixture or cross linked poly(acrylate) ester because the control was not formulated with water. Significant mass loss was observed for the three step transitions between 65° and 200° C. followed by relatively constant mass for the three step transitions between 200° and 350° C. Significant mass loss did not occur until the 350° to 450° C. step transition which we have interpreted as decomposition and volatilization of the actual cross linked polymer.

### Calculations

1. The exclusion of mass loss below 65° C. as either adsorbed or absorbed water within the fragrance/IPM/polymer matrix

2. Interpretation of volatile mass loss within the 65-350° C. thermal range as fragrance/IPM mixture (A)

3. Interpretation of volatile mass loss within the 350-450° C. thermal range as decomposition of cross linked poly(acrylate) ester (B)

4. Summation of A, B and C and normalization to 100% mass loss

5. Summation of A and C divided by 100 to calculate the fragrance/IPM fraction

6. Division of B by 100 to calculate the cross linked poly(acrylate) ester fraction after normalization to 100% mass loss.

TABLE 2

| | Thermal Range (C. °) | | | | | | | |
|---|---|---|---|---|---|---|---|---|
| | 25-65 | 25-450 | 65-350 | 350-450 | >450 | | | |
| | 0-60 | 0-250 | 60-225 | 225-250 | Corrected | | Percent | Percent |
| | Minute | Minute | Minute | Minute | Non- | | Volatile | Volatile |
| Description | Volatiles | Volatiles | Volatiles | Volatiles | volatiles | Total | 65-350° C. | 350-450° C. |
| Water Control | 98.5 | | | | | | | |
| Reference | | 96.4 | | | | | | |
| perfume oil | | | | | | | | |
| Perfume | 28.5 | | 63.9 | 5.3 | 2.3 | 71.5 | 92.4 | 7.6 |
| Microcapsule | | | | | | | | |
| Slurry CP1341 | | | | | | | | |

For example, this particular perfume microcapsule slurry has 7.6% Percent Coating of the Microcapsule Shell.

### Test Method for Determining of the Percentage Overall Mass of the Shell (for Both Coated or Uncoated Microcapsules)

From the thermal gravimetric analysis method presented above, the overall mass of the shell can be obtained by multiplying the Percent Coating of the Microcapsule Shell by the total mass of the microcapsule. For example in 1 gram of microcapsule with a 7.6% coating of the shell, there would be 0.076 grams of shell material.

### Test Method for Determining the Core to Shell Mass Ratio

From the thermal gravimetric method presented above, the core to shell mass ratio is determined by percent volatiles (65-350 C) and percent volatiles 350 C-450 C. In the example presented in Table 2, the core to shell mass ratio is 92.4 to 7.6.

### Test Method for Determining Shell Thickness

One skilled in the art will recognize that various protocols may be constructed for the extraction and isolation of microcapsules from finished products, and will recognize that such methods require validation via a comparison of the resulting measured values, as measured before and after the microcapsules' addition to and extraction from the finished product. The isolated microcapsules are then formulated in DI water to form a slurry for characterization.

A Cryo-SEM is utilized to characterize the morphology of the microcapsules and measure the average wall thickness of particles. Each specimen is plunge frozen into liquid ethane, then transferred to the Gatan Alto cryo-prep chamber while maintaining temperatures below −170° C. The samples are equilibrated at −130° C., then sliced, then immediately coated with Au/Pd for about 70 s. Imaging is performed on the Hitachi 4700, or equivalent, at 3 KV and 20 μA tip current at −140° C. The shell thickness is reported as a range.

### Dispersibility Test Method

1. For each slurry containing microcapsules to be tested, prepare one VWR Spatula with PVC Handle (Item #82027-502) by ensuring the PVC handle is clean, smooth, and dust-free.

2. Fully submerge the PVC handle of the spatula into the melted composition until the composition fully covers the PVC handle (not the blade end).

3. Hold PVC handle submerged in composition for period of 10 seconds.

4. Remove PVC handle and hold over composition for 10 seconds, allowing any residual composition to drip off.

5. Place spatula on paper towel or other substrate for drying. Allow 1 minute to dry.

6. Once dry, inspect PVC handle to ensure microcapsules are substantially fully dispersed within the composition. This is done visually by confirming that the composition is smooth and uniform on the PVC handle, with an absence of any crevices, specks, unevenness, coarseness, protrusions , or otherwise, lack of uniformity. Presence of aggregates indicates microcapsules are not sufficiently dispersed in the composition.

7. Repeat for all compositions.

### Glass Transition Temperature Measurement Method

One skilled in the art will recognize that various protocols may be constructed for the extraction and isolation of microcapsules from finished products, and will recognize that such methods require validation via a comparison of the resulting measured values, as measured before and after the microcapsules' addition to and extraction from the finished product. The isolated microcapsules are then formulated in DI water to form a slurry for characterization.

The glass transition temperature is measured using Differential Scanning calorimetry (DSC): ASTM E1356, “Standard Test Method for Assignment of the Glass Transition Temperature by Differential Scanning calorimetry” described below.

The normal operating temperature range is from −120 to 500 ° C. The temperature range may be extended, depending upon the instrumentation used. The values stated in SI units are to be regarded as standard. No other units of measurement are included in this standard. The following terms are applicable to this test method and can be found in Terminology E473 and Terminology E1142: differential scanning calorimetry (DSC); differential thermal analysis (DTA); glass transition; glass transition temperature (T_{g}); and specific heat capacity. Definitions of Terms Specific to This Standard: There are commonly used transition points associated with the glass transition region:

extrapolated end temperature, (Tₑ), ° C.—the point of intersection of the tangent drawn at the point of greatest slope on the transition curve with the extrapolated baseline following the transition.
extrapolated onset temperature, (T_{f}), ° C.—the point of intersection of the tangent drawn at the point of greatest slope on the transition curve with the extrapolated baseline prior to the transition.
inflection temperature, (Tᵢ), ° C.—the point on the thermal curve corresponding to the peak of the first derivative (with respect to time) of the parent thermal curve. This point corresponds to the inflection point of the parent thermal curve.
midpoint temperature, (Tₘ), ° C.—the point on the thermal curve corresponding to ½ the heat flow difference between the extrapolated onset and extrapolated end.
Discussion—Midpoint temperature is most commonly used as the glass transition temperature.
Two additional transition points are sometimes identified and are defined:
temperature of first deviation, (Tₒ), ° C.—the point of first detectable deviation from the extrapolated baseline prior to the transition.
Temperature of return to baseline, (Tᵣ), ° C.—the point of last deviation from the extrapolated baseline beyond the transition.

A change in heating rates and cooling rates can affect the results. The presence of impurities will affect the transition, particularly if an impurity tends to plasticize or form solid solutions, or is miscible in the post-transition phase. If particle size has an effect upon the detected transition temperature, the specimens to be compared should be of the same particle size.

In some cases the specimen may react with air during the temperature program causing an incorrect transition to be measured. Whenever this effect may be present, the test shall be run under either vacuum or an inert gas atmosphere. Since some materials degrade near the glass transition region, care must be taken to distinguish between degradation and glass transition.

Since milligram quantities of sample are used, it is essential to ensure that specimens are homogeneous and representative, so that appropriate sampling techniques are used.

Differential Scanning calorimeter, The essential instrumentation required to provide the minimum differential scanning calorimetric capability for this method includes a Test Chamber composed of a furnace(s) to provide uniform controlled heating (cooling) of a specimen and reference to a constant temperature or at a constant rate over the temperature range from −120 to 500° C., a temperature sensor to provide an indication of the specimen temperature to 60.1° C., differential sensors to detect heat flow difference between the specimen and reference with a sensitivity of 6 μW, a means of sustaining a test chamber environment of a purge gas of 10 to 100 mL/min within 4 mL/min, a Temperature Controller, capable of executing a specific temperature program by operating the furnace(s) between selected temperature limits at a rate of temperature change of up to 20° C./min constant to 6 0.5° C./min.

### Apparatus

Differential Scanning calorimeter, The essential instrumentation required to provide the minimum differential scanning calorimetric capability for this method includes a Tes tChamber composed of a furnace(s) to provide uniform controlled heating (cooling) of a specimen and reference to a constant temperature or at a constant rate over the temperature range from −120 to 500° C., a temperature sensor to provide an indication of the specimen temperature to 60.1° C., differential sensors to detect heat flow difference between the specimen and reference with a sensitivity of 6 μW, a means of sustaining a test chamber environment of a purge gas of 10 to 100 mL/min within 4 mL/min, a Temperature Controller, capable of executing a specific temperature program by operating the furnace(s) between selected temperature limits at a rate of temperature change of up to 20° C./min constant to 6 0.5° C./min.

A Data Collection Device, To provide a means of acquiring, storing, and displaying measured or calculated signals, or both. The minimum output signals required for DSC are heat flow, temperature and time.

Containers, (pans, crucibles, vials, etc.) that are inert to the specimen and reference materials and that are of suitable structural shape and integrity to contain the specimen and references.

For ease of interpretation, an inert reference material with an heat capacity approximately equivalent to that of the specimen may be used. The inert reference material may often be an empty specimen capsule or tube.

Nitrogen, or other inert purge gas supply, of purity equal to or greater than 99.9%.

Analytical Balance, with a capacity greater than 100 mg, capable of weighing to the nearest 0.01 mg.

### Specimen Preparation

Powders or Granules—Avoid grinding if a preliminary thermal cycle as outlined in 10.2 is not performed. Grinding or similar techniques for size reduction often introduce thermal effects because of friction or orientation, or both, and thereby change the thermal history of the specimen.

Molded Parts or Pellets—Cut the samples with a microtome, razor blade, paper punch, or cork borer (size No. 2 or 3) to appropriate size in thickness or diameter, and length that will approximate the desired mass in the subsequent procedure.

For thinner films, cut slivers to fit in the specimen tubes or punch disks, if circular specimen pans are used.—For films thicker than 40 μm, see “Molded Parts or Pellets”.

### Calibration

Using the same heating rate, purge gas, and flow rate as that to be used for analyzing the specimen, calibrate the temperature axis of the instrument following the procedure given in Practice E967.

### Procedure

10.1 Use a specimen mass appropriate for the material to be tested. In most cases a 5 to 20 mg mass is satisfactory. An amount of reference material with a heat capacity closely matched to that of the specimen may be used. An empty specimen pan may also be adequate.

10.2 If appropriate, perform and record an initial thermal program in flowing nitrogen or air environment using a heating rate of 10° C./min to a temperature at least 20° C. above Tₑ to remove any previous thermal history. (See FIG. 1.)

NOTE 1—Other, preferably inert, gases may be used, and other heating and cooling rates may be used, but must be reported.

10.3 Hold temperature until an equilibrium as indicated by the instrument response is achieved.

10.4 Program cool at a rate of 20° C./min to 50° C. below the transition temperature of interest.

10.5 Hold temperature until an equilibrium as indicated by the instrument response is achieved.

10.6 Repeat heating at same rate as in 10.2, and record the heating curve until all desired transitions have been completed. Other heating rates may be used but must be reported.

10.7 Determine temperatures Tₘ (preferred) T_{f}, or Tᵢ, where:
Tig=inflection temperature, ° C.
Tf=extrapolated onset temperature, ° C., and
Tm=midpoint temperature, ° C.

Increasing the heating rate produces greater baseline shifts thereby improving detectability.

In the case of DSC the signal is directly proportional to the heating rate in heat capacity measurements.

10.8 Recheck the specimen mass to ensure that no loss or decomposition has occurred during the measurement.

### Fracture Strength Test Method

One skilled in the art will recognize that various protocols may be constructed for the extraction and isolation of microcapsules from finished products, and will recognize that such methods require validation via a comparison of the resulting measured values, as measured before and after the microcapsules' addition to and extraction from the finished product. The isolated microcapsules are then formulated in DI water to form a slurry for characterization.

To calculate the percentage of microcapsules which fall within a claimed range of fracture strengths, three different measurements are made and two resulting graphs are utilized. The three separate measurements are namely: i) the volume-weighted particle size distribution (PSD) of the microcapsules; ii) the diameter of at least 10 individual microcapsules within each of 3 specified size ranges, and; iii) the rupture-force of those same 30 or more individual microcapsules. The two graphs created are namely: a plot of the volume-weighted particle size distribution data collected at i) above; and a plot of the modeled distribution of the relationship between microcapsule diameter and fracture-strength, derived from the data collected at ii) and iii) above. The modeled relationship plot enables the microcapsules within a claimed strength range to be identified as a specific region under the volume-weighted PSD curve, and then calculated as a percentage of the total area under the curve.
a) The volume-weighted particle size distribution (PSD) of the microcapsules is determined via single-particle optical sensing (SPOS), also called optical particle counting (OPC), using the AccuSizer 780 AD instrument, or equivalent, and the accompanying software CW788 version 1.82 (Particle Sizing Systems, Santa Barbara, Calif., U.S.A.). The instrument is configured with the following conditions and selections: Flow Rate=1 ml/sec; Lower Size Threshold=0.50 μm; Sensor Model Number=LE400-05SE; Autodilution=On; Collection time=120 sec; Number channels=512; Vessel fluid volume=50 ml; Max coincidence=9200. The measurement is initiated by putting the sensor into a cold state by flushing with water until background counts are less than 100. A capsule slurry, and its density of particles is adjusted with DI water as necessary via autodilution to result in particle counts of at least 9200 per ml. During a time period of 120 seconds the suspension is analyzed. The resulting volume-weighted PSD data are plotted and recorded, and the values of the mean, 5^{th} percentile, and 90^{th} percentile are determined.
b) The diameter and the rupture-force value (also known as the bursting-force value) of individual microcapsules are measured via a computer-controlled micromanipulation instrument system which possesses lenses and cameras able to image the microcapsules, and which possesses a fine, flat-ended probe connected to a force-transducer (such as the Model 403A available from Aurora Scientific Inc, Canada, or equivalent), as described in: Zhang, Z. et al. (1999) “Mechanical strength of single microcapsules determined by a novel micromanipulation technique.” J. Microencapsulation, vol 16, no. 1, pages 117-124, and in: Sun, G. and Zhang, Z. (2001) “Mechanical Properties of Melamine-Formaldehyde microcapsules.” J. Microencapsulation, vol 18, no. 5, pages 593-602, and as available at the University of Birmingham, Edgbaston, Birmingham, UK.
c) A drop of the microcapsule suspension is placed onto a glass microscope slide, and dried under ambient conditions for several minutes to remove the water and achieve a sparse, single layer of solitary particles on the dry slide. Adjust the concentration of microcapsules in the suspension as needed to achieve a suitable particle density on the slide. More than one slide preparation may be needed.
d) The slide is then placed on a sample-holding stage of the micromanipulation instrument. Thirty or more microcapsules on the slide(s) are selected for measurement, such that there are at least ten microcapsules selected within each of three pre-determined size bands. Each size band refers to the diameter of the microcapsules as derived from the Accusizer-generated volume-weighted PSD. The three size bands of particles are: the Mean Diameter +/−2 μm; the 5^{th} Percentile Diameter +/−2 μm; and the 90^{th} Percentile Diameter +/−2 μm. Microcapsules which appear deflated, leaking or damaged are excluded from the selection process and are not measured.
e) For each of the 30 or more selected microcapsules, the diameter of the microcapsule is measured from the image on the micromanipulator and recorded. That same microcapsule is then compressed between two flat surfaces, namely the flat-ended force probe and the glass microscope slide, at a speed of 2 μm per second, until the microcapsule is ruptured. During the compression step, the probe force is continuously measured and recorded by the data acquisition system of the micromanipulation instrument.
f) The cross-sectional area is calculated for each of the microcapsules, using the diameter measured and assuming a spherical particle (πr², where r is the radius of the particle before compression). The rupture force is determined for each sample by reviewing the recorded force probe measurements. The measurement probe measures the force as a function of distance compressed. At one compression, the microcapsule ruptures and the measured force will abruptly stop. This maxima in the measured force is the rupture force.
g) The Fracture Strength of each of the 30 or more microcapsules is calculated by dividing the rupture force (in Newtons) by the calculated cross-sectional area of the respective microcapsule.
h) On a plot of microcapsule diameter versus fracture-strength, a Power Regression trend-line is fit against all 30 or more raw data points, to create a modeled distribution of the relationship between microcapsule diameter and fracture-strength.
i) The percentage of microcapsules which have a fracture strength value within a specific strength range is determined by viewing the modeled relationship plot to locate where the curve intersects the relevant fracture-strength limits, then reading off the microcapsule size limits corresponding with those strength limits These microcapsule size limits are then located on the volume-weighted PSD plot and thus identify an area under the PSD curve which corresponds to the portion of microcapsules falling within the specified strength range.

The identified area under the PSD curve is then calculated as a percentage of the total area under the PSD curve. This percentage indicates the percentage of microcapsules falling with the specified range of fracture strengths.

### Extraction Method to Analyze % Total Perfume Loading of a Microcapsule

One skilled in the art will recognize that various protocols may be constructed for the extraction and isolation of microcapsules from finished products, and will recognize that such methods require validation via a comparison of the resulting measured values, as measured before and after the microcapsules' addition to and extraction from the finished product. The isolated microcapsules are then formulated in DI water to form a slurry for characterization.

Weigh and record weight of 30 mg of PMC (i.e. perfume microcapsule) slurry. Add 20 mL of Internal Standard solution (25 mg/L Dodecane in anhydrous alcohol) and heat at 60° C. for 30 minutes. Cool to room temperature. Filter through 0.45 μm PTFE syringe filter. Analyze via GC/FID.

### Instruments Used:

Agilent 6890NGC/FID
Agilent 7683B Injector
Balance:
Column: J&W DB-5 (20 m×0.1 mm×0.1 um)

### Instrument Conditions:

GC Conditions
Oven: 50° C. for 0 minute; Ramp at 16° C./minute to 275° C., hold 3 minutes
Inlet Split mode: Temp: 250° C.; Split ratio 80:1; Flow: 0.4 mL/minute; Inj volume: 1 μL

FID Conditions
325° C.; Hydrogen: 40 mL/minute; Make-up 25 mL/minute; Air: 400 mL/minute

### Data Analysis:

% Encapsulated=(((STD Perfume Conc./Area (perf std))×(ISTD Area (perf std)/ISTD Area (sample))×AREA (sample))/Sample Conc.)×100%

### Hexane Extraction Test Method

0.10 g of PMC powder is preweighed in a 50 mL vial

10 mL of hexane is added to the vial

The sample is vortexed for 20 seconds

The sample is shaken using an automated hand shaker for 10 minutes

The sample is allowed to sit at room temperature for 10 minutes to allow for phase separation

The hexane layer is filtered through a 0.45 micrometer PTFE filter

The filtered material is injected into a GC/MS to analyze the components extracted

The GC/MS trace of the sample is compared to a control. The control is prepared using neat perfume (unencapsulated) in hexane based on the % of the total perfume loading of the capsule obtained using the method above. The ratio of the total fragrance amount in the extracted sample to the control allows one to calculate the free oil (unencapsulated oil) in the powder sample.

### Process Yield Test Method

Measure the % solids concentration of perfume microcapsule slurry (using the Microwave method described herein). Record the mass of perfume microcapsule slurry that is spray dried. Record the mass of perfume microcapsule spray dried powder collected, with an inlet air temperature of 205 degrees Centigrade and outlet air temperature of 105 degrees Centigrade. Divide the mass of spray dried powder collected by the mass of perfume microcapsule slurry dried multiplied by the wt % solids concentration of the slurry. This is the process yield.

### Bulk Flow Energy Test Method

Use the FT4 Powder Rheometer (available from Freeman Technology Inc., Medford, N.J., USA), to determine powder flowability. Prepare Assembly that will hold the spray dried powder (per FT4 instructions). Tare the assembly. Add powder. Accept/Record the mass. Close the lid. Begin the split. The screw will insert into the sample to condition the sample. After conditioning is complete, open the lid of the powder rheometer, and then do a split (this removes excess powder above the container), and the instrument is now ready to analyze the bulk flow properties of the powder. Let test run on its own (8 tests run at a tip speed of 100 millimeters/second—the screw will go into and out of the sample). Recover sample, and clean the instrument with a brush.

### Microwave Method

1) Measure the % solids concentration of perfume microcapsule slurry (i.e. capsule slurry)
a. Supplies and Materials
   i. CEM Oven—CEM Smart System 5 (available from CEM Corporation, Matthews, N.C. USA)
   ii. Sample pads—CEM square pads, item #200150
   iii. Transfer pipette
1.1 Vigorously shake capsule slurry until homogenous (The capsule batch should be mixed well and not separated).
1.2 Press MAIN MENU button.

1.3 Press 3—LOAD METHOD.
1.4 Press number of applicable method.
   1.4.1 (example: PHOENIX50)
1.5 Press the arrow button to select Solids or Moisture.
1.6 Press READY.
1.7 Open lid of oven and tare 2 pieces of square sample pads by pressing TARE. (See FIG. 2)
1.8 Remove the top square pad.
1.9 Using a pipet, put a zigzag line of slurry onto the remaining pad, enough to equal about 1.5 grams. (See FIG. 3). Use the side of the pipet to spread it across the pad.
1.10 Replace the top square sample pad.
1.11 Close lid.
1.12 Press START.
1.13 When finished, lift hood and remove sample. Record results on sample container.
1.14 Close lid.
1.15 Clean up any spills.
1.16 Processing will take anywhere from 5-15 minutes. Oven will beep when it is finished and produce a printout. The printout will list: Time/date, Method used, Sample # (just a numeric number that is given), Drying time, Max. temp., Initial weight, and % solids/moisture.

### EXAMPLES

A perfume composition, called Scent A, is utilized to prepare the examples of the invention. The table below lists the ingredients, and their properties.

TABLE 1

| Material Name | ClogP | Boiling Point ° C. |
|---|---|---|
| Beta Gamma Hexenol | 1.3 | 155 |
| Phenyl Ethyl Alcohol | 1.32 | 219 |
| Helional | 1.77 | 329 |
| Triplal Extra | 1.78 | 199 |
| Amyl-Acetate (isomer Blends) | 1.87 | 135 |
| Melonal | 2.09 | 182 |
| Liffarome | 2.14 | 167 |
| Iso Eugenol Acetate | 2.17 | 303 |
| Cis 3 Hexenyl Acetate | 2.18 | 167 |
| Jasmolactone | 2.36 | 219 |
| 2′6-nonadien-1-ol | 2.43 | 213 |
| Florosa | 2.46 | 238 |
| Nonalactone | 2.66 | 193 |
| Cis Jasmone | 2.81 | 254 |
| Ethyl Linalool | 2.92 | 223 |
| Pino Acetaldehyde | 2.98 | 261 |
| Methyl Dihydro Jasmonate | 3.01 | 323 |
| Undecavertol | 3.06 | 242 |
| Azurone 10/tec 0015573 | 3.06 | 395 |
| Dihydro Myrcenol | 3.08 | 195 |
| Cyclemax | 3.23 | 281 |
| Hivernal | 3.29 | 351 |
| Pomarose | 3.51 | 214 |
| Undecalactone | 3.75 | 228 |
| Damascenone Total 937459 | 3.89 | 267 |
| Acalea (01-1963) | 3.9 | 344 |
| Cis-3-hexenyl Salicylate | 4 | 316 |
| Ionone Beta | 4.02 | 267 |
| Polysantol | 4.21 | 256 |
| Ambroxan | 4.58 | 285 |
| 5-cyclohexadecen-1-one | 5.04 | 331 |
| Iso E Super Or Wood | 5.05 | 325 |
| Laevo Muscone | 5.48 | 321 |
| Helvetolide 947650 | 5.56 | 309 |

### Example 1

### Nonionic Microcapsules

An oil solution, consisting of 75 g Fragrance Oil scent A, 75 g of Isopropyl Myristate, 0.6 g DuPont Vazo-52, and 0.4 g DuPont Vazo-67, is added to a 35° C. temperature controlled steel jacketed reactor, with mixing at 1000 rpm (4 tip, 2″ diameter, flat mill blade) and a nitrogen blanket applied at 100 cc/min. The oil solution is heated to 75° C. in 45 minutes, held at 75° C. for 45 minutes, and cooled to 60° C. in 75 minutes.

A second oil solution, consisting of 37.5 g Fragrance Oil, 0.25 g tertiarybutylaminoethyl methacrylate, 0.2 g 2-carboxyethyl acrylate, and 10 g Sartomer CN975 (hexafunctional urethane-acrylate oligomer) is added when the first oil solution reached 60° C. The combined oils are held at 60° C. for an additional 10 minutes.

Mixing is stopped and a water solution, consisting of 56 g of 5% active polyvinyl alcohol Celvol 540 solution in water, 244 g water, 1.1 g 20% NaOH, and 1.2 g DuPont Vazo-68WSP, is added to the bottom of the oil solution, using a funnel.

Mixing is again started, at 2500 rpm, for 60 minutes to emulsify the oil phase into the water solution. After milling is completed, mixing is continued with a 3″ propeller at 350 rpm. The batch is held at 60° C. for 45 minutes, the temperature is increased to 75° C. in 30 minutes, held at 75° C. for 4 hours, heated to 90° C. in 30 minutes and held at 90° C. for 8 hours. The batch is then allowed to cool to room temperature forming a microcapsule slurry. The finished microcapsules have a median particle size of 11 microns, a broadness index of 1.3, and a zeta potential of negative 0.5 millivolts, and a total scent A concentration of 19.5 wt %, and a water content of 57 wt %.

### Example 2

### Conventional Spray Drying of Perfume Microcapsules

The perfume microcapsule slurry of Example 1 is pumped at a rate of 7.7 g/min into a co-current spray dryer (Buchi, 10 inch diameter) and atomized using a 2 fluid nozzle (40100 SS nozzle, 1250 air cap). Dryer operating conditions are: air flow of 600 Liters per minute, an inlet air temperature of 185 degrees Centigrade, an outlet temperature of 85 degrees Centigrade, dryer operating at a pressure of −30 millibar, atomizing air pressure of 100 psi. The dried powder is collected at the bottom of a cyclone and under the dryer (oversize). The collected particles have an approximate particle diameter of 11 microns. Approximately 17.5 grams of powder is collected, resulting in a yield of 20%. A significant amount of product coats the chamber wall. A separate run greater than 1 hour results in significant reduction in powder yield because the powder forms a bridge across the chamber, restricting air flow and reducing the volume available to dry the atomized particle. A Differential Scanning calorimeter is used to measure the glass transition temperature of the spray dried powder. It is found that the onset of the glass transition occurs around 82 degrees Centigrade, with the final glass transition temperature of approximately 108 degrees centigrade. The equipment used for the spray drying process may be obtained from the following suppliers: IKA Werke GmbH & Co. KG, Janke and Kunkel—Str. 10, D79219 Staufen, Germany; Niro A/S Gladsaxevej 305, P.O. Box 45, 2860 Soeborg, Denmark and Watson-Marlow Bredel Pumps Limited, Falmouth, Cornwall, TR11 4RU, England.

### Example 3

### Spray Drying of Perfume Microcapsules with Particulates

To the perfume microcapsule slurry of Example 1 is added various process aids in order to improve product yield. For clarity, 1.5% colloidal silica in Capsule Slurry means that the enough colloidal silica is transferred to the capsule slurry so that the colloidal silica constitutes 1.5% by weight of the capsule slurry after addition to the capsule slurry. Table 3A provides details on the process aids used, their composition in the perfume microcapsule slurry, and the product yield.

TABLE 3A

| | | | | | 3% Colloidal | | | |
|---|---|---|---|---|---|---|---|---|
| | | 1.5% | | 6% | Silica in | | | 1.5% |
| | | colloidal | 3% colloidal | colloidal | Capsule | | | Precipitated |
| | | silica in | silica in | silica in | Slurry & | | | Silicon |
| | | Capsule | Capsule | Capsule | Higher Outlet | | 3% Sodium | Dioxide in |
| | | Slurry | Slurry | Slurry | Air | | Montmorillonite | Capsule |
| | No | (Ludox HS- | (Ludox HS- | (Ludox HS- | Temperature | 3% Talc | in Capsule | Slurry |
| | Process | 30 Process | 30 Process | 30 Process | (Ludox HS-30 | in Capsule | Slurry | (Sipernat |
| Description of Sample | Aid | Aid) | Aid) | Aid) | Process Aid) | Slurry | (Bentonite) | 22S) |
| Example | 2 | 3A | 3B | 3C | 3D | 3E | 3F | 3G |
| Microcapsules of Example 1 | 200 | 448 | 430 | 395 | 430 | 455 | 455 | 460 |
| Process Aid (g) | 0 | 25 | 50 | 100 | 50 | 15 | 15 | 7.5 |
| Water (g) | 20.8 | 27 | 20 | 5 | 20 | 30 | 30 | 33 |
| Inlet Air Temp © | 185 | 185 | 185 | 185 | 200 | 185 | Not dried | 185 |
| Outlet Air Temp © | 85 | 85 | 85 | 85 | 105 | 85 | because | 85 |
| Feed Rate (pump) | 35 | 42 | 42 | 58 | 25 | 40 | Bentonite does | 65 |
| Atomizing Air (psi) | 100 | 100 | 100 | 100 | 100 | 100 | not disperse | 100 |
| Air Flow (L/min) | 600 | 600 | 600 | 600 | 600 | 600 | well in the | 600 |
| Aspirator % | 100 | 100 | 100 | 100 | 100 | 100 | slury-large | 100 |
| Chamber Vacuum (mbar) | −30 | −30 | −30 | −30 | −30 | −30 | aggregates that | −30 |
| Time to Dry (min) | 26 | 58 | 62 | 43 | 107 | 13 | could not be | 18 |
| Cyclone Collector (g) | 17.8 | 95.7 | 97 | 107.7 | 113.5 | 9.5 | broken up even | 19.8 |
| Oversize (g) | 0 | 14.9 | 15.8 | 10.9 | 17.8 | 0 | with intense | 0 |
| Bowl (g) | N/A | 39.6 | 41.5 | 40.2 | 15.3 | N/A | mixing. | N/A |
| % Yield (Cyclone) | 22% | 48% | 49% | 54% | 57% | 24% | | 45% |
| % Yield (Cyclone + Oversize) | 22% | 55% | 56% | 59% | 66% | 24% | | 45% |
| g/min water dried | 4.62 | 5.17 | 4.84 | 6.98 | 2.80 | 4.62 | | 3.67 |
| g/min product | 0.68 | 1.91 | 1.82 | 2.76 | 1.23 | 0.73 | | 1.10 |
| % lost as fines | N/A | 25% | 23% | 21% | 27% | N/A | | N/A |

Note that the addition of colloidal silica as a process aid significantly improves the product yield. The mixture of perfume microcapsule slurry and the process aid is pumped into a co-current spray dryer (Buchi, 10 inch diameter) and atomized using a 2 fluid nozzle (40100 SS nozzle, 1250 air cap). Dryer operating conditions are itemized in Table 3A. The dried powder is collected at the bottom of a cyclone and at the bottom of the dryer (oversize). The collected particles have an approximate particle diameter of 11 microns. The equipment used for the spray drying process may be obtained from the following suppliers: IKA Werke GmbH & Co. KG, Janke and Kunkel—Str. 10, D79219 Staufen, Germany; Niro A/S Gladsaxevej 305, P.O. Box 45, 2860 Soeborg, Denmark and Watson-Marlow Bredel Pumps Limited, Falmouth, Cornwall, TR11 4RU, England.

Micrographs of some of the spray-dried microcapsules are shown in FIGS. 8-10, indicating that the colloidal silica particles coat the perfume microcapsule, but these particles do not provide a hermetic coating to the microcapsules. As a result, we do not change the mechanical properties of the microcapsules.

FIG. 8 is a micrograph showing spray dried uncoated microcapsules 817A.

FIG. 9 is a micrograph showing spray dried microcapsules 817B partially coated with particulates 849, from a 1.5% Ludox HS-30 process aid in the slurry, as described above.

FIG. 10 is a micrograph showing spray dried microcapsules 817C partially coated with particulates 849, from a 3% Ludox HS-30 process aid in the slurry, as described above.

### Example 4

### Spray Dried Macrocapsules

To 94.85 kilograms of nonionic perfume microcapsule made by the method of example 1 is added 0.15 kilograms of Xanthan Gum powder (Novaxan Dispersible Xanthan Gum Product 174965) at a temperature of 45 degrees Centigrade, while mixing. After 25 minutes of mixing, 4.5 kilograms of a 32 wt % solution of magnesium chloride is added to the slurry (over a period of 10 minutes), then the slurry is mixed for an additional 30 minutes. An appropriate preservative system is added to the slurry to control micro susceptibility. Next, 1 kilogram of citric acid (anhydrous powder) is added, and mixed for 30 minutes to assure complete dissolution in the continuous phase of the slurry. This mixture is then atomized using a co-current Niro dryer, 7 ft diameter, using a rotary centrifugal wheel atomizer The specific drying conditions are captured in Table 4A.

TABLE 4A

| Description | Example 4W | Example 4X | Example 4Y |
|---|---|---|---|
| Inlet Air Temp ° C. | 195 | 218 | 232 |
| Outlet Air Temp ° C. | 85 | 107 | 116 |
| Feed Solids % | 35% | 35% | 35% |
| % Yield | less than 20% | 75% | 82% |
| Moisture % | 6.1% | 5.1% | 4.7% |
| Bulk Flow Energy (mJ) | Not Measured | 383 | 448 |
| Bulk Density (g/L) | Not Measured | 380 | 408 |
| Free Oil % | 13% | 11% | 10% |
| (unencapsulated oil) | | | |

Note that when the outlet air temperature of the working fluid is close to or below the glass transition temperature of the microcapsules (Example 4W), a very low process yield is obtained, and the recovered microcapsules have a high level of unencapsulated oil. When the operating temperature of the working fluid is at or above the glass transition temperature Example 4X, 4Y), the process yield increases dramatically, and the unencapsulated oil is also lower.

### Example 5

### Microcapsules in Antiperspirant/Deodorant

TABLE 5A

| | Example | Example | Example | Example | Example |
|---|---|---|---|---|---|
| Ingredient | I | II⁹ | III | IV | V |
| Part I: Partial | | | | | |
| Continuous | | | | | |
| Phase | | | | | |
| Hexamethyl- | 22.65 | 21.25 | 21.25 | 21.25 | 21.25 |
| disiloxane¹ | | | | | |
| DC5200² | 1.20 | 1.20 | 1.20 | 1.20 | |
| Fragrance | 0.35 | 1.25 | 1.25 | 1.25 | 1.25 |
| Fragrance | 1.00 | 1.00 | 1.00 | 1.00 | 1.00 |
| Capsules | | | | | |
| of Example 3 | | | | | |
| Shin Etsu KF | | | | | 1.20 |
| 6038³ | | | | | |
| Part II: Disperse | | | | | |
| Phase | | | | | |
| ACH (40% | 40.00 | 55.0 | | | |
| solution)⁴ | | | | | |
| IACH (34% | | 2.30 | 49.00 | | |
| solution)⁵ | | | | | |
| ZAG (30% | | | | 52.30 | 52.30 |
| solution)⁶ | | | | | |
| propylene | 5.00 | | 5.00 | 5.00 | 5.00 |
| glycol | | | | | |
| Water | 12.30 | | 3.30 | | |
| Part III: | | | | | |
| Structurant Plus | | | | | |
| Remainder of | | | | | |
| Continuous | | | | | |
| Phase | | | | | |
| FinSolve TN | 6.50 | 6.00 | 6.50 | 6.00 | 6.50 |
| Ozokerite Wax | | | | | 12.00 |
| Performalene | 11.00 | 11.00 | 12.00 | 12.00 | |
| PL⁷ | | | | | |
| Aqueous Phase | 37.7 | 79.5 | 40.5 | 60.3 | 60.3 |
| Conductivity | | | | | |
| (mS/cm) | | | | | |

¹DC 246 fluid from Dow Coming
²from Dow Corning
³from Shinetsu
⁴Standard aluminum chlorohydrate solution
⁵IACH solution stabilized with calcium
⁶IZAG solution stabilized with calcium
⁷from New Phase Technologies
⁹emulsion broke when manufacturing this composition

The above examples I through V can be made via the following general process, which one skilled in the art will be able to alter to incorporate available equipment. The ingredients of Part I and Part II are mixed in separate suitable containers. Part II is then added slowly to Part I under agitation to assure the making of a water-in-silicone emulsion. The emulsion is then milled with suitable mill, for example a Greeco 1L03 from Greeco Corp, to create a homogenous emulsion. Part III is mixed and heated to 88° C. until the all solids are completely melted. The emulsion is then also heated to 88° C. and then added to the Part 3 ingredients. The final mixture is then poured into an appropriate container, and allowed to solidify and cool to ambient temperature.

TABLE 5B

| Ingredient | VI | VII | VIII | IX | X |
|---|---|---|---|---|---|
| Product Form | Solid | Solid | Solid | Solid | Deo- |
| | Deo- | Deo- | Deo- | Deo- | dorant |
| | dorant | dorant | dorant | dorant | or Body |
| | | | | | Spray |
| dipropylene | 45 | 22 | 20 | 30 | 20 |
| glycol | | | | | |
| propylene | 22 | 45 | 22 | | |
| glycol | | | | | |
| tripopylene | | | 25 | | |
| glycol | | | | | |
| Glycerine | | | | 10 | |
| PEG-8 | | | | 20 | |
| ethanol | | | | | QS |
| Water | QS | QS | QS | QS | |
| sodium stearate | 5.5 | 5.5 | 5.5 | 5.5 | |
| tetra sodium | 0.05 | 0.05 | 0.05 | 0.05 | |
| EDTA | | | | | |
| sodium | 0.04 | 0.04 | 0.04 | 0.04 | |
| hydroxide | | | | | |
| triclosan | 0.3 | 0.3 | 0.3 | 0.3 | |
| Fragrance | 0.5 | 0.5 | 0.5 | 0.5 | 0.5 |
| Fragrance | 1.0 | 1.0 | 1.0 | 1.0 | 0.5 |
| capsules of | | | | | |
| Example 3 | | | | | |
| dihydro- | 0.3 | .1 | 0.3 | 0.5 | .1 |
| myrcenol | | | | | |
| Linalool | 0.2 | .15 | 0.2 | 0.25 | .15 |
| Propellant (1,1 | | | | | 40 |
| difluoroethane) | | | | | |

QS—indicates that this material is used to bring the total to 100%.

Examples VI to IX can be made as follows: all ingredients except the fragrance, linalool, and dihydromyrcenol are combined in a suitable container and heated to about 85° C. to form a homogenous liquid. The solution is then cooled to about 62° C. and then the fragrance, linalool, and dihydromyrcenol are added. The mixture is then poured into an appropriate container and allowed to set up while cooling to ambient temperature.

Example X can be made as follows: all the ingredients except the propellant are combined in an appropriate aerosol container. The container is then sealed with an appropriate aerosol delivery valve. Next air in the container is removed by applying a vacuum to the valve and then propellant is added to container through the valve. Finally an appropriate actuator is connected to the valve to allow dispensing of the product.

TABLE 5C

| | Example XI | Example XII | Example XIII |
|---|---|---|---|
| | Invisible Solid | Invisible Solid | Soft Solid |
| Aluminum Zirconium | 24 | 24 | 26.5 |
| Trichlorohydrex | | | |
| Glycine Powder | | | |
| Cyclopentasiloxane | Q.S | Q.S. | Q.S. |
| Dimethicone | — | — | 5 |
| CO-1897 Stearyl | 14 | 14 | — |
| Alcohol NF | | | |
| Hydrogenated Castor | 3.85 | 3.85 | — |
| Oil MP80 Deodorized | | | |
| Behenyl Alcohol | 0.2 | 0.2 | — |
| Tribehenin | — | — | 4.5 |
| C 18-36 acid | — | — | 1.125 |
| triglyceride | | | |
| C12-15 Alkyl | 9.5 | 9.5 | — |
| Benzoate | | | |
| PPG-14 Butyl Ether | 6.5 | 6.5 | 0.5 |
| Phenyl Trimethicone | 3 | — | — |
| White Petrolatum | — | 3 | 3 |
| Mineral Oil | 1.0 | 1.0 | — |
| Fragrance | 0.75 | 0.75 | 0.75 |
| Talc Imperial 250 | 3.0 | 3.0 | — |
| USP | | | |
| Fragrance Capsules | 1.9 | 1.5 | 1.75 |
| of Example 3 | | | |

QS—indicates that this material is used to bring the total to 100%.

### Example 6

### Dry Laundry Detergent Composition

Non-limiting examples of product formulations containing purified perfume microcapsules of the aforementioned examples are summarized in the following table.

TABLE 6

| | % w/w granular laundry detergent | | | | | | |
|---|---|---|---|---|---|---|---|
| | composition | | | | | | |
| Component | A | B | C | D | E | F | G |
| Brightener | 0.1 | 0.1 | 0.1 | 0.2 | 0.1 | 0.2 | 0.1 |
| Soap | 0.6 | 0.6 | 0.6 | 0.6 | 0.6 | 0.6 | 0.6 |
| Ethylenediamine | 0.1 | 0.1 | 0.1 | 0.1 | 0.1 | 0.1 | 0.1 |
| disuccinic acid | | | | | | | |
| Acrylate/maleate | 1.5 | 1.5 | 1.5 | 1.5 | 1.5 | 1.5 | 1.5 |
| copolymer | | | | | | | |
| Hydroxyethane | 0.4 | 0.4 | 0.4 | 0.4 | 0.4 | 0.4 | 0.4 |
| di(methylene | | | | | | | |
| phosphonic acid) | | | | | | | |
| Mono-C₁₂₋C₁₄ alkyl, | 0.5 | 0.5 | 0.5 | 0.5 | 0.5 | 0.5 | 0.5 |
| di-methyl, mono- | | | | | | | |
| hydroyethyl | | | | | | | |
| quaternary | | | | | | | |
| ammonium chloride | | | | | | | |
| Linear alkyl benzene | 0.1 | 0.1 | 0.2 | 0.1 | 0.1 | 0.2 | 0.1 |
| Linear alkyl benzene | 10.3 | 10.1 | 19.9 | 14.7 | 10.3 | 17 | 10.5 |
| sulphonate | | | | | | | |
| Magnesium sulphate | 0.4 | 0.4 | 0.4 | 0.4 | 0.4 | 0.4 | 0.4 |
| Sodium carbonate | 19.5 | 19.2 | 10.1 | 18.5 | 29.9 | 10.1 | 16.8 |
| Sodium sulphate | 29.6 | 29.8 | 38.8 | 15.1 | 24.4 | 19.7 | 19.1 |
| Sodium Chloride | 0.1 | 0.1 | 0.1 | 0.1 | 0.1 | 0.1 | 0.1 |
| Zeolite | 9.6 | 9.4 | 8.1 | 18 | 10 | 13.2 | 17.3 |
| Photobleach particle | 0.1 | 0.1 | 0.2 | 0.1 | 0.2 | 0.1 | 0.2 |
| Blue and red | 1.8 | 1.8 | 1.8 | 1.8 | 1.8 | 1.8 | 1.8 |
| carbonate speckles | | | | | | | |
| Ethoxylated | 1 | 1 | 1 | 1 | 1 | 1 | 1 |
| Alcohol AE7 | | | | | | | |
| Tetraacetyl ethylene | 0.9 | 0.9 | 0.9 | 0.9 | 0.9 | 0.9 | 0.9 |
| diamine agglomerate | | | | | | | |
| (92 wt % active) | | | | | | | |
| Citric acid | 1.4 | 1.4 | 1.4 | 1.4 | 1.4 | 1.4 | 1.4 |
| PDMS/clay | 10.5 | 10.3 | 5 | 15 | 5.1 | 7.3 | 10.2 |
| agglomerates | | | | | | | |
| (9.5% wt % | | | | | | | |
| active PDMS) | | | | | | | |
| Polyethylene oxide | 0.2 | 0.2 | 0.2 | 0.2 | 0.2 | 0.2 | 0.2 |
| Enzymes e.g. Protease | 0.2 | 0.3 | 0.2 | 0.1 | 0.2 | 0.1 | 0.2 |
| (84 mg/g active), | | | | | | | |
| Amylase | | | | | | | |
| (22 mg/g active) | | | | | | | |
| Suds suppressor | 0.2 | 0.2 | 0.2 | 0.2 | 0.2 | 0.2 | 0.2 |
| agglomerate | | | | | | | |
| (12.4 wt % active) | | | | | | | |
| Sodium percarbonate | 7.2 | 7.1 | 4.9 | 5.4 | 6.9 | 19.3 | 13.1 |
| (having from 12% to | | | | | | | |
| 15% | | | | | | | |
| active AvOx) | | | | | | | |
| Perfume oil | 0.5 | 0.5 | 0.5 | 0.5 | 0.5 | 0.5 | 0.5 |
| Solid perfume | 0.4 | 0 | 0.4 | 0.4 | 0.4 | 0.4 | 0.6 |
| particles | | | | | | | |
| Perfume | 1.3 | 2.4 | 1 | 1.3 | 1.3 | 1.3 | 0.7 |
| microcapsules* | | | | | | | |
| Water | 1.4 | 1.4 | 1.4 | 1.4 | 1.4 | 1.4 | 1.4 |
| Misc | 0.1 | 0.1 | 0.1 | 0.1 | 0.1 | 0.1 | 0.1 |
| Total Parts | 100 | 100 | 100 | 100 | 100 | 100 | 100 |

*Microcapsule added as powder or agglomerate. Core/wall ratio can range from 80/20 up to 98/2 and average particle diameter can range from 5 μm to 50 μm. Suitable combinations of the microcapsules provided in Examples 2, 3 and 4.

### Example 7

### Perfume Microcapsules in Unit Dose Formulations

The following are examples of unit dose executions wherein the liquid composition is enclosed within a PVA film. The preferred film used in the present examples is Monosol M8630 76 μm thickness. The preference is to incorporate the dry microcapsules with the dry powders; however, since these formulations are typically low water (due to the sensitivity of polyvinyl alcohol to water), the microcapsules can be incorporated into either the liquid or powder containing compartments.

TABLE 7

| | D | | | E | | F | | |
|---|---|---|---|---|---|---|---|---|
| | 3 | | | 2 | | 3 | | |
| | compartments | | | compartments | | compartments | | |
| Compartment # | 42 | 43 | 44 | 45 | 46 | 47 | 48 | 49 |
| Dosage (g) | 34.0 | 3.5 | 3.5 | 30.0 | 5.0 | 25.0 | 1.5 | 4.0 |
| Ingredients | Weight % | | | | | | | |
| Alkylbenzene sulfonic acid | 20.0 | 20.0 | 20.0 | 10.0 | 20.0 | 20.0 | 25 | 30 |
| Alkyl sulfate | | | | 2.0 | | | | |
| C₁₂₋₁₄ alkyl 7-ethoxylate | 17.0 | 17.0 | 17.0 | | 17.0 | 17.0 | 15 | 10 |
| C₁₂₋₁₄ alkyl ethoxy 3 sulfate | 7.5 | 7.5 | 7.5 | | | 7.5 | 7.5 | |
| Citric acid | 0.5 | | 2.0 | 1.0 | | | | 2.0 |
| Zeolite A | | | | 10.0 | | | | |
| C₁₂₋₁₈ Fatty acid | 13.0 | 13.0 | 13.0 | | 18.0 | 18.0 | 10 | 15 |
| Sodium citrate | | | | 4.0 | 2.5 | | | |
| Enzymes | 0-3 | 0-3 | 0-3 | 0-3 | | 0-3 | 0-3 | 0-3 |
| Sodium Percarbonate | | | | 11.0 | | | | |
| TAED | | | | 4.0 | | | | |
| Polycarboxylate | | | | 1.0 | | | | |
| Ethoxylated Polyethylenimine¹ | 2.2 | 2.2 | 2.2 | | | | | |
| Hydroxyethane diphosphonic acid | 0.6 | 0.6 | 0.6 | 0.5 | | | 2.2 | |
| Ethylene diamine tetra(methylene | | | | | | 0.4 | | |
| phosphonic) acid | | | | | | | | |
| Brightener | 0.2 | 0.2 | 0.2 | 0.3 | | 0.3 | | |
| Microcapsules* | 0.4 | 1.2 | 1.5 | 1.3 | 1.3 | 0.4 | 0.12 | 0.2 |
| Water | 9 | 8.5 | 10 | 5 | 11 | 10 | 10 | 9 |
| CaCl2 | | | | | | | 0.01 | |
| Perfume | 1.7 | 1.7 | | 0.6 | | 1.5 | 0.5 | |
| Minors (antioxidant, sulfite, | 2.0 | 2.0 | 2.0 | 4.0 | 1.5 | 2.2 | 2.2 | 2.0 |
| aesthetics) | | | | | | | | |

| | |
|---|---|
| Buffers (sodium carbonate, | To pH 8.0 for liquids |
| monoethanolamine) ³ | To RA > 5.0 for powders |
| Solvents (1,2 propanediol, | To 100 p |
| ethanol), Sulfate | |

¹Polyethylenimine (MW = 600) with 20 ethoxylate groups per —NH.
² RA = Reserve Alkalinity (g NaOH/dose)
*Microcapsule added as 25-35% active slurry (aqueous solution, example 1) or as a spray dried powder (Example 2 and 3). Core/wall ratio can range from 80/20 up to 98/2 and average particle diameter can range from 5 μm to 50 μm. Suitable combinations of the microcapsules provided in Examples 1 through 3.
** Low water liquid detergent in Polyvinylalcohol unidose/sachet

### Example 8

### Addition of Powder to Thick Substrate

The following surfactant/polymer liquid processing composition is prepared at the indicated weight percentages as described in Table 8 below.

TABLE 8A

| Component | |
|---|---|
| Glycerin | 3.2 |
| Polyvinyl alcohol¹ | 8.1 |
| Sodium Lauroamphoacetate (26% activity)² | 31.8 |
| Ammonium Laureth-3 sulfate (25% activity) | 4.9 |
| Ammonium Undecyl sulfate (24% activity) | 19.9 |
| Ammonium Laureth-1 sulfate (70% activity) | 8.0 |
| Cationic cellulose³ | 0.5 |
| Citric Acid | 1.6 |
| Distilled water | 22.0 |
| Total | 100.0 |
| pH | 5.8 |
| Viscosity (cp) | 35,400 |

¹Sigma-Aldrich Catalog No. 363081, MW 85,000-124,000, 87-89% hydrolyzed
²McIntyre Group Ltd, University Park, IL, Mackam HPL-28ULS
³UCARE ™ Polymer LR-400, available from Amerchol Corporation (Plaquemine, Louisiana)

A target weight of 300 grams of the above composition is prepared with the use of a conventional overhead stirrer (IKA® RW20DZM Stirrer available from IKA® Works, Inc., Wilmington, Del.) and a hot plate (Corning Incorporated Life Sciences, Lowell, Mass.). Into an appropriately sized and cleaned vessel, the distilled water and glycerin are added with stirring at 100-150 rpm. The cationic polymer, when present, is then slowly added with constant stirring until homogenous. The polyvinyl alcohol is weighed into a suitable container and slowly added to the main mixture in small increments using a spatula while continuing to stir while avoiding the formation of visible lumps. The mixing speed is adjusted to minimize foam formation. The mixture is slowly heated to 80° C. after which surfactants are added. The mixture is then heated to 85° C. while continuing to stir and then allowed to cool to room temperature. Additional distilled water is added to compensate for water lost to evaporation (based on the original tare weight of the container). The final pH is between 5.2-6.6 and adjusted with citric acid or diluted sodium hydroxide if necessary. The resulting processing mixture viscosity is measured.

A porous dissolvable solid substrate (also referred to in the examples herein as “substrate”) is prepared from the above liquid processing mixture as described in Table 8 below.

TABLE 8B

| | |
|---|---|
| Aeration Time (sec) | 62 |
| Wet Density (g/cm³) | 0.26 |
| Oven Temperature (° C.) | 130 |
| Drying Time (min) | 38 |
| Average dry substrate weight (g) | 1.10 |
| Average dry substrate thickness (cm) | 0.62 |
| Average substrate shrinkage (%) | 4.6% |
| Average dry substrate density (g/cm³) | 0.11 |
| Average basis weight (g/m²) | 650 |

300 grams of the processing mixture is stored within a convection oven for greater than two hours at 70° C. to pre-heat the processing mixture. The mixture is then transferred into a pre-heated 5 quart stainless steel bowl (by placing into 70° C. oven for greater than 15 minutes) of a KITCHENAID® Mixer Model K5SS (available from Hobart Corporation, Troy, Ohio) fitted with a flat beater attachment and with a water bath attachment comprising tap water at 70-75° C. The mixture is vigorously aerated at a maximum speed setting of 10 until a wet density of approximately 0.26 grams/cm³ is achieved (time recorded in table). The density is measured by weighing a filling a cup with a known volume and evenly scraping off the top of the cup with a spatula. The resulting aerated mixture is then spread with a spatula into square 160 mm×160 mm aluminum molds with a depth of 6.5 mm with the excess wet foam being removed with the straight edge of a large metal spatula that is held at a 45° angle and slowly dragged uniformly across the mold surface. The aluminum molds are then placed into a 130° C. convection oven for approximately 35 to 45 minutes. The molds are allowed to cool to room temperature with the substantially dry porous dissolvable solid substrates removed from the molds with the aid of a thin spatula and tweezers.

Each of the resulting 160 mm×160 mm square substrates is cut into nine 43 mm×43 mm squares (with rounded edges) using a cutting die and a Samco SB20 cutting machine (each square representing surface area of approximately 16.9 cm²). The resulting smaller substrates are then equilibrated overnight (14 hours) in a constant environment room kept at 70° F. and 50% relative humidity within large zip-lock bags that are left open to the room atmosphere.

Within a fume hood, the substrate is mounted on a stainless steel easel that rests at about a 60 degree angle and with notches holding the substrate from sliding downward and with a hole in plate so that the substrate can easily be removed from the mount by pushing from the easel. It is important that the top surface of the substrate (the side that is exposed to the air in the drying oven and opposite the side that is in direct contact with the aluminum mold during the drying process) is facing away from the easel. A small glass bottle with a pump spray is filled with the primary fragrance oil 1a and then sprayed onto the surface of the substrate from a distance of 2 to 3 inches. The substrate is then removed from the easel and returned to the weigh boat on the balance with the top side facing upwards. The weight of perfume applied is recorded and in the instance that the target weight is not achieved, either another spray amount is applied or a Kim wipe to absorb excess perfume away from the substrate. This iterative process is repeated until the target weight range is achieved. The amount of fragrance la applied is recorded in the below table. The resulting substrate resting on the small weigh boat is stored within a zip-lock bag and sealed from the atmosphere. The above process is repeated on a second substrate.

The first substrate within its weigh boat is later removed from the zip-lock bag and tared again to zero weight on a 4 place weigh balance. A perfume microcapsule of Example 2 and 3 is then applied to the surface of each substrate. The substrate is coated with the perfume microcapsule powder by gently shaking the substrate in a tray (or other suitable container) containing an excess of the perfume inclusion complex in a side-to-side manner ten times (the process is repeated for the other side). The resulting powder coated substrate is then picked up (with gloved hands) and gently shaken and tapped several times to remove any excess powder that is not sufficiently adhered to the substrate. The resulting weight of the microcapsule of the secondary fragrance applied is recorded in the below table. The porous substrate within its weigh boat is then returned the zip lock bag and sealed from the atmosphere. This powder application process is repeated for the second substrate.

The final weights achieved are given in the below table:

TABLE 8C

| | | | Weight of Scent |
|---|---|---|---|
| | Initial | | A perfume |
| | substrate | Weight of primary | microcapsule powder |
| Substrate No. | weight | fragrance applied | (Example 21) |
| 1 | 1.194 | 0.050 | 0.0175 |
| 2 | 1.063 | 0.055 | 0.0150 |
| Averages | 1.129 | 0.053 | 0.0161 |

### Example 9

### Dry Shampoo Powder Composition

Perfume microcapsules of Example 2 and 3 can be mixed with other powders that formulate a dry shampoo product. Such powders can have the following composition:

TABLE 9A

| Material | A | B | C | D | E | F |
|---|---|---|---|---|---|---|
| Tapioca Starch | 55.2% | 64.0% | 76.4% | 38.7% | 54.8% | 53.7% |
| Talc Powder | 27.6% | 32.0% | 12.7% | 38.7% | 27.4% | 26.8% |
| Bentonite Powder | 6.9% | 0.0% | 6.4% | 12.9% | 6.8% | 6.7% |
| Aerosil 200 | 2.8% | 3.2% | 2.5% | 2.6% | 2.7% | 2.7% |
| Magnesium | 6.9% | 0.0% | 1.3% | 6.5% | 6.8% | 6.7% |
| Stearate | | | | | | |
| Perfume | 0.7% | 0.8% | 0.6% | 0.6% | 1.4% | 3.4% |
| Microcapsule | | | | | | |

Tapioca starch is available from Akzo Nobel, Talc powder and bentonite powder can be purchased from Kobo Products, Aerosil 200 can be obtained from Evonik Degussa corporation, Magnesium stearate can be obtained from Sigma Aldrich.

### Example 10

### Nonwoven

Perfume microcapsules can be incorporated during the process of making a nonwoven.

### Example 11

### Spray Drying of Perfume Microcapsules with Particulates for High Yields of Spray-Dried Microcapsules

Add To 1000 grams of the perfume microcapsule slurry of Example 1 (43% solids), approximately 43 grams of a 30 wt % suspension of Ludox HS-30 colloidal silica. This slurry is then pumped at a rate of 7.7 g/min into a co-current spray dryer (Buchi, 10 inch diameter) and atomized using a 2 fluid nozzle (40100 SS nozzle, 1250 air cap). Dryer operating conditions are: air flow of 600 Liters per minute, an inlet air temperature of 200 degrees Centigrade, an outlet temperature of 102 degrees Centigrade, dryer operating at a pressure of −30 millibar, atomizing air pressure of 100 psi. The dried powder is collected at the bottom of a cyclone and under the dryer (oversize). The collected microcapsules have an approximate diameter of 11 microns. Approximately 410 grams of powder is collected, resulting in a yield of 95%. The equipment used for the spray drying process may be obtained from the following suppliers: IKA Werke GmbH & Co. KG, Janke and Kunkel—Str. 10, D79219 Staufen, Germany; Niro A/S Gladsaxevej 305, P.O. Box 45, 2860 Soeborg, Denmark and Watson-Marlow Bredel Pumps Limited, Falmouth, Cornwall, TR11 4RU, England.

The values disclosed herein are not to be understood as being strictly limited to the exact numerical values recited. Instead, unless otherwise specified, each value such is intended to mean both the recited value and a functionally equivalent range surrounding that value. For example, a median volume-weighted particle size disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
a plurality of microcapsules comprising a core material and a shell encapsulating the core material; and
an adjunct ingredient;
wherein the microcapsules have a median volume-weighted average particle size of from 3 micrometers to 25 micrometers;
wherein the shell of the microcapsules are coated with particulates.

2. The composition of claim 1, wherein the shell comprises a polyacrylate material.

3. The composition of claim 1, wherein the shell comprises a polyacrylate material having a total polyacrylate mass and including material selected from the group consisting of: amine content of from 0.2% to 2.0% of the total polyacrylate mass; carboxylic acid of from 0.6% to 6.0% of the total polyacrylate mass; and a combination of amine content of from 0.1% to 1.0% and carboxylic acid of from 0.3% to 3.0% of the total polyacrylate mass.

4. The composition of claim 1, wherein the shell has a thickness of from 1 nanometer to 300 nanometers.

5. The composition of claim 1, wherein the particulates have a median volume-weighted particle size of from 1 nanometer to 1000 nanometers.

6. The composition of claim 1, wherein the particulates comprise inorganic particulates.

7. The composition of claim 1, wherein the particulates comprise silica particulates.

8. The composition of claim 1, wherein the particulates are selected from the group consisting of precipitated silicas, colloidal silicas, fumed silicas, and mixtures thereof.

9. The composition of claim 1, wherein the particulates comprise material selected from the group consisting of citric acid, sodium carbonate, sodium sulfate, magnesium chloride, potassium chloride, sodium chloride, sodium silicate, modified cellulose, zeolite, silicon dioxide, and combinations thereof.

10. The composition of claim 1, wherein the core material has a first mass and the shell has a second mass, wherein the ratio of the first mass to the second mass is 80% to 20%

11. The composition of claims 1, wherein from 15% to 85% of the shell of the microcapsules is coated with the particulates.

12. The composition of claim 1, wherein the microcapsules have a bulk flow energy of from 1 milliJoule to 800 milliJoules, according to the Bulk Flow Energy Test Method.

13. The composition of claim 1, wherein the shell of the microcapsules is coated with the particulates using a spray-drying process.

14. The composition of claim 1, wherein the microcapsules have a fracture strength of from 0.2 mega Pascals to 10.0 mega Pascals, according to the Fracture Strength Test Method.

15. A method of making a composition comprising:
spray-drying a plurality of microcapsules with a plurality of particulates to form a plurality of spray-dried microcapsules; and
adding the plurality of the spray-dried microcapsules to an adjunct ingredient to form a composition;
wherein the microcapsules comprise a core material and a shell encapsulating the core material; wherein the spray-dried microcapsules comprise the core material and the shell encapsulating the core material; wherein the spray-dried microcapsules are coated with the particulates.

16. The method of claim 15, wherein the method further comprises:
providing an aqueous slurry comprising the microcapsules; and
providing a colloidal suspension comprising the particulates;
wherein the spray-drying includes spraying-drying the aqueous slurry and the colloidal suspension.

17. The method of claim 15, wherein:
the shell has a glass transition temperature that is less than or equal to a first temperature;
and the spray-drying includes spray-drying the microcapsules with a working fluid;
wherein the working fluid is at a temperature that is greater than the first temperature;
wherein the first temperature is from 75 degrees Celsius to 150 degrees Celsius;
wherein the working fluid is heated to a temperature that is from 25 degrees Celsius to 175 degrees Celsius greater than the first temperature.

18. The method of claim 15, wherein from 15% to 85%, of the shell of the spray-dried microcapsules is coated with the particulates.

19. The method of claim 15, wherein the spray-dried microcapsules have a bulk flow energy of from 1 milliJoule to 800 milliJoules, according to the Bulk Flow Energy Test Method.

20. The method of claim 15, wherein the method produces a process yield of greater than 60% but less than or equal to 95% of the spray-dried microcapsules, according to the Process Yield Test Method.
